(19) United States
(12) Patent Application Publication
(10) Pub. No.: US 2014/0303109 A1
(43) Pub. Date: Oct. 9, 2014

(54) Novel Analogs of Curcumin and Methods of Use

(71) Applicants: Fazlul H. Sarkar, Plymouth, MI (US); Subhash Padhye, Pune (IN)

(72) Inventors: Fazlul H. Sarkar, Plymouth, MI (US); Subhash Padhye, Pune (IN)

(21) Appl. No.: 13/637,358

(22) PCT Filed: Mar. 25, 2011
(86) PCT No.: PCT/US11/00555

§ 371 Date: Oct. 21, 2013

Related U.S. Application Data
(60) Provisional application No. 61/341,186, filed on Mar. 25, 2010.

(51) Int. Cl. C07F 1/08; A61K 31/12; A61K 31/7068; A61K 31/282; A61K 31/353; A61K 31/415; A61K 31/513; C07C 49/255; A61K 31/30

(52) U.S. Cl. CPC C07F 1/08; C07C 49/255; A61K 31/12; A61K 31/7068; A61K 31/30; A61K 31/353; A61K 31/415; A61K 31/513; A61K 31/282
USPC 514/49; 568/325; 514/679; 556/117; 514/499; 514/500; 514/456; 514/406; 514/274; 514/492

(57) ABSTRACT

Water-soluble fluorinated analogs of natural curcumin, and particularly difluoro Knoevenagel condensates and Schiff bases, along with their corresponding copper (H) complexes have improved bioavailablity over curcumin. The fluorine-substituted analogs of curcumin are useful as chemopreventive and/or therapeutic agents against cancers and/or against the development of drug-resistant cancer. A preferred compound is (IE,6E)-1,7-bis(4-hydroxy-3-methoxyphenyl)hepta-1,6-diene{4(3,4 difluorobenzaldehyde)}-3,5-dione.

### CROSS-REFERENCE TO RELATED INVENTIONS

This application claims the benefit of priority under 35 U.S.C. §119(e) of U.S. provisional application Ser. No. 61/341,186 filed on Mar. 25, 2010, the disclosure of which is incorporated herein by reference.

### GOVERNMENT RIGHTS

This invention was made with Government support by the National Institutes of Health Contract Numbers R01CA083695, R01CA131151, and R01CA132794.

### BACKGROUND OF THE INVENTION

1. Field of the Invention

This invention relates generally to novel analogs of curcumin, and more particularly, to water-soluble fluorinated analogs of curcumin, that are useful, inter alia, as anti-cancer agents, and methods of treating cancers, particularly colon cancer and pancreatic cancer, to prevent drug-resistance and/or potentiate the effects of known chemotherapeutic agents.

2. Description of the Prior Art

Curcumin, an active phenolic compound extracted from the rhizome of the plant Curcuma longa, has long been used as a spice (tumeric) and coloring agent in Indian cuisine and as a therapeutic agent in traditional Indian Ayurvedic Medicine for treating a variety of health disorders, including respiratory conditions, liver disorders, inflammation, diabetic wounds, coughs, and certain tumors. Recent investigations have provided evidence that curcumin does, indeed, prevent a variety of carcinogen-induced cancers and suppresses the mutagenic effects of various carcinogens, including tobacco and cigarette smoke condensates.

Naturally-occurring curcumin exhibits anti-cancer activities both in vitro and in vivo through a variety of mechanisms, although these mechanisms are stilly not fully understood at this time. Curcumin has been shown to inhibit proliferation and induce apoptosis in a wide variety of cancer cells, including bladder, breast, lung, pancreas, prostate, cervix, head and neck, ovary, kidney, brain and skin, through interaction with numerous biochemicals and molecular targets (e.g., transcription factors, growth factors and their receptors, cytokines, enzymes) either through direct interaction, or through modulation of gene expression.

Curcumin has also been found to potentiate the effects of some known therapeutic agents, already in clinical use, such as genistein, celecoxib, gemcitabine, 5-flurouracil and oxaliplatin.

Advantageously, curcumin is non-toxic and safe. It does not cause any adverse effects in humans, even in doses as high as 8 gm per day. Furthermore, there have been no reports to date of the development of resistance against the effects of curcumin.

The bioavailability of curcumin, however, limits its therapeutic utility. As much as 75% of administered curcumin is rapidly metabolized and conjugated in the liver, and gets excreted in the feces indicating poor absorption from the gut. As a result, the bioavailability is limited. When injected intravenously, the majority of the drug is metabolized and actively transported into bile, suggesting that curcumin has poor absorption, but rapid metabolism. There have been various attempts to improve the bioavailability of curcumin, including the co-administration of piperine, a known inhibitor of hepatic and intestinal glucuronidation, and the use of various drug delivery systems, including liposomes, micelles, phospholipid complexes and nanoparticles. However, these attempts at improving the bioavailability of curcumin, or slowing down metabolism, have been largely unsuccessful.

Curcumin is a symmetrical β-diketone. Referring to FIG. 6A, two aromatic rings containing phenolic groups are connected by two α,β-unsaturated carbonyl groups. The carbonyl groups form a diketone which exists in keto- and enol-tautomeric forms. The energetically more stable enol form is known to exist predominately in acidic and neutral solutions, as well as in cell membranes. The enol form can be deprotonated easily under mild alkaline conditions. This facile tautomeric conversion may contribute to the rapid metabolism of curcumin. In an effort to slow down the rapid metabolism of curcumin, Zambre, et al., Copper Conjugates of Knoevenagel Condensates of Curcumin and their Schiff Base Derivatives: Synthesis, Spectroscopy, Magnetism, ESR, and Electrochemistry, Synthesis and Reactivity in Inorganic, Metal-Organic, and Nano-Metal Chemistry, Vol. 37, pages 19-27, (2007) blocked the tautomeric conversion by Knoevenagel condensation of the active methylenic group of curcumin.

Another strategy that has been investigated to improve the biological activity of curcumin is through its complexation with metal ions. See, for example, Anand, et al., Biological Activities of Curcumin and its Analogues (Congeners) Made by Man and Mother Nature, Biochem. Pharmacol., Vol. 76, pages 1590-1611 (2008); and Anand, et al., Bioavailability of curcumin: problems and promises, Mol. Pharm., Vol. 4, pages 807-818 (2007). Anti-tumor activity has been reported for such metal complexes by John, et al., Antitumor studies of metal chelates of synthetic curcuminoids, J. Exp. Clin. Cancer Res., Vol. 21, pages 219-224 (2002). However, none of these strategies have yielded compounds that have better anti-tumor activity than curcumin.

In order to obtain compounds that may have better anti-tumor activity than curcumin, as well as improved bioavailability, there have been attempts in the prior art to produce analogs of the naturally-occurring curcumin. The known structural modifications made to the curcumin molecule have been summarized in Padhye, et al., Perspective on Chemopreventive and Therapeutic Potential of Curcumin Analogs in Medicinal Chemistry, Mini-Reviews in Medicinal Chemistry, Vol. 10, No. 5, pages 372-387 (2010). Referring to the curcumin molecule on FIG. 6A, these structural changes include placing substituents on the aryl side chains appended to the β-diketone at position 1, modifying the conjugate double bond (between positions 1 and 2), modifying the diketo functionality, and modifying the carbon adjacent to the double bond (positions 1 and 7). Some of these analogs have been shown to exhibit potent effects against cancer cells, and are, in some cases, more potent than the parent curcumin. While the studies relating to the modified structures have provided useful insight into the mechanism of action of curcumin, and its analogs, the results with respect to improved serum or tissue bioavailabity, have been disappointing.

It is, therefore, an object of this invention to provide novel analogs of naturally-occurring curcumin that have better anti-tumor activity, along with increased bioavailability, and hence, improved potential for use as chemo-preventive and/or therapeutic agents.

As indicated above, curcumin interacts with many biochemicals and molecular targets because it affects several cellular receptors (EGFR and HER2), signal transcription factors (NF-κB, AP-1, Egr-1, β-catenin, and PPAR-γ), various oxygenases, such as COX-2 and 5-lipoxygenase (5-LOX), inducible nitric oxide synthase (iNOS), cell cycle proteins (cyclin D1 and p21), cytokines (TNF, IL-1, IL-6, chemokines), as well as cell surface adhesion molecules. See, Aggarwal, et al., Curcumin: the Indian solid gold, Adv. Exp. Med. Biol., Vol. 595, pages 1-75 (2007). The studies reported by Aggarwal, et al. clearly suggest that curcumin might be useful for the prevention and/or treatment of human cancers. There have been many reports linking inflammatory mediators, including cytokines, e.g., TNF-alpha, IL-6, IL-8 and interferon-gamma; transcription factors such as NF-κB; and proinflammatory enzymes, like cyclooxygenase, as well as lipooxygenase isoforms, with the development and progression of pancreatic cancer (PC). The expression of COX-2 has been found to be increased in a variety of malignancies, including PC. It is now well-established that COX-2-mediated synthesis of prostaglandins (PGE₂) favors the growth of tumor cells by stimulating proliferation and angiogenesis, while over-expression of COX-2 inhibits apoptosis. COX-2 expression is regulated, in part, by a transcriptional mechanism mediated by the transcription factor NFκB. The effects of curcumin have been shown to be mediated by inactivation of NFκB, leading to the conclusion that curcumin might be useful to inhibit pancreatic cancer progression.

Although significant progress has been made in systemic treatments, pancreatic cancer still remains the fourth leading cause of cancer-related deaths in the United States. The high mortality rate is due, in large part, to the high incidence of metastatic disease at initial diagnosis, the aggressive nature of PC tumors, and the lack of effective systemic therapies. As a result, the disease-free survival time, even after complete surgical resection of tumorous tissue, and adjuvant administration of a cytotoxic agent, such as gemcitabine, is less than a year.

Gemcitabine is considered to be the standard agent for the treatment of the advanced pancreatic cancer, and has offered some relief over the past two decades. However, attempts to improve the survival of patients diagnosed with pancreatic cancer, such as by combination treatments to improve the effectiveness of gemcitibine, have been disappointing. There is, therefore, a need for better treatment strategies to improve the survival outcome of pancreatic cancer patients.

There is emerging evidence that combination therapy, involving inclusion of curcumin, may increase the effectiveness of conventional therapies. For example, the combination of curcumin and gemcitabine has been shown to have an inhibitory effect on PC cell lines. Curcumin in combination with celecoxib, a COX-2 inhibitor, showed significant growth inhibition of PC cell lines and, interestingly, in combination with omega-3 fatty acids, showed synergistic tumor inhibitory properties. These results suggest that curcumin could be useful in combination therapy with conventional agents, particularly in view of the fact that curcumin is non-toxic to humans and has shown multi-targeted effects. Furthermore, curcumin alone can alter the expression of microRNAs in PC cells, which could be important in mediating its biological effects.

Although curcumin can inhibit cell viability and induce apoptosis in pancreatic, breast, lung, prostate and several other cancer cell lines, and is well-tolerated, its limited bioavailability has limited its therapeutic value, especially for the treatment of patients with pancreatic tumors. Numerous analogs of curcumin have been created to overcome its low bioavailability and to increase its absorption without loss of activity, however none of these analogs has shown better target tissue bioavailability, especially in the pancreas. There is, thus, a need for analogs of curcumin that have increased bioavailability and greater targeting to the pancreas.

It is, therefore, a further object of this invention to provide analogs of naturally-occurring curcumin that have increased bioavailability, and in particular, improved target tissue bioavailabilty in the pancreas.

Colorectal carcinoma is even more prevalent than pancreatic cancer. The present most widely used chemotherapeutic agents for colon cancer are 5-Fluorouracil (5-FU) or a combination of 5-fluorouracil and oxaliplatin (5-FU+Ox), or FOLFOX, which further includes folinic acid. The response to these agents, however, is often incomplete. Nearly 50% of patients with colorectal carcinoma cancer, treated by conventional therapeutics, will have a recurrence.

Although the reasons for recurrence are not fully understood, a growing body of evidence suggests that it could due, at least in part, to enrichment of chemotherapy-resistant cancer stem/stem-like cells (CSCs) that retain a limitless potential to regenerate. Of course, pancreatic cancer is also subject to acquired resistance to conventional therapies, including chemotherapy, such as gemcitibine, alone or in combination with other cytotoxic or targeted agents, and radiation. In addition to the capacity for self-renewal, CSCs have the potential to regenerate into all types of differentiated cells, giving rise to heterogeneous tumor cell populations in a tumor mass which contribute to tumor aggressiveness. There is, thus, a need for a therapeutic agent and/or method of treatment that reduces and/or eliminates CSCs in tumors, including, colon and pancreatic cancer tumors, thereby improving the treatment outcome and survival rate. There is further a need for a therapeutic agent and/or method of treatment that selectively targets CSCs, but spares normal stem cells that may rely on similar mechanisms of action for self-renewal.

Of course, continued use of chemotherapy can lead to additional toxicities, some of which may be fatal. Therefore, is therefore, a need for non-toxic therapeutic agents and/or methods of treatment that do not cause the development of resistance.

Curcumin has been reported to synergize the combination treatment 5-FU+Ox, and to inhibit the growth of colon cancer cells, and in particular, colon CSCs, in vitro. Various independent studies have shown that the combination treatment of curcumin with a variety of chemotherapy drugs, including cisplatin, danorubicin, doxorubicin, and vinscristine, enhances the cellular accumulation of these drugs, and thereby increases the sensitivity to these chemotherapeutics. Again, the use of curcumin as a therapeutic agent has been hampered by its poor bioavailability.

Thus, there is a need for a therapeutic agent that is non-toxic like curcumin, but that has better bioavailabity, that can potentiate the action of known chemotherapeutic agents, without increasing toxicity or causing the cancer cells to develop resistance.

### SUMMARY OF THE INVENTION

The foregoing and other objects are achieved by this invention which provides novel fluorinated analogs of curcumin, and more particularly fluorinated Knoevenagel condensates of curcumin and corresponding Schiff bases, along with their copper (II) complexes, that have greater bioavilability than curcumin (CUR) and known CUR analogs. The analogs of the present invention are non-toxic and have improved bioavailability and anti-cancer activity.

In one aspect of the invention, novel curcumin analogs in accordance with the invention have the general Formula I:

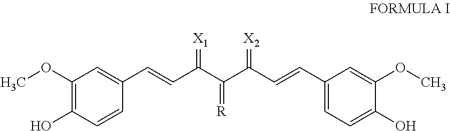

wherein X₁ and X₂ at the 3,5 positions of the 1,5-diaryl pentadienone are the same or different, and are selected from the group consisting of O, —OH, or an amino.

The dashed lines indicate an optional double bond depending on whether the curcumin analog is in the keto or enol form. In preferred embodiments, X₁═X₂═O.

The amino substituent may be an alkylamine, arylamine, heterocyclic amine, phenylamine, such as aniline, naphthoylamine, isothiocyanate, semicarbazide, thiosemicarbazide, hydrazone, hydrazide, thiourea, hydroxamate, arylazo, azocylic, carboxyamidrazone, or the like.

In preferred compounds, X₁ and/or X₂, and preferably X₁═X₂, is thiosemicarbazide or semicarbazide of the general formula:

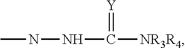

where Y═O or S and R₃ and R₄ are selected from the group of —H and substituted or unsubstituted aryl, phenyl, alkyl, or aralykyl substituents.

In preferred embodiments, the analog has a difluorinated substituent at the active methylene 4-position of the 1,5-diaryl pentadienone. In particularly preferred embodiments, R a fluorinated benzyl of the general formula:

—CHₙ-[*insert phenyl with x₁-x₅ substituents]

wherein x₁-x₅ are selected from the group consisting of —H, —F, —CF₃, —CHF₂, and CHₙ is a saturated or unsaturated lower alkyl where n=1-4.

In a particularly preferred embodiment of the invention, the curcumin analog has a fluorine-containing substituent at the 4-position, which is 3,4 difluorobenzyl, as shown in Formula II below:

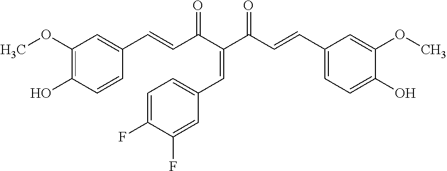

The compound of Formula II has the chemical name: (1E,6E)-1,7-bis(4-hydroxy-3-methoxyphenyl)hepta-1,6-diene{4(3,4 difluorobenzaldehyde)}-3,5-dione (herein coded as CDF; FIG. 1, Compound 2). CDF is a Knoevenagle condensate that can be reacted with a nitrogen-containing reactant, such as an amine, or a hydrazide, to prepare 3,5-disubstituted Schiff Bases. Both the Knoevenagle condensate and the Schiff Base form ligands that will conjugate with a metal ion, which in preferred embodiments is a Cu(II) ion.

The following compound is the copper conjugate of the condensate CDF named (1E,6E)-1,7-bis(4-hydroxy-3-methoxyphenyl)hepta-1,6-diene{4(3,4 difluorobenzaldehyde)}-3,5-dione Cu(II) coded herein as CDFCu (FIG. 1, Compound 7).

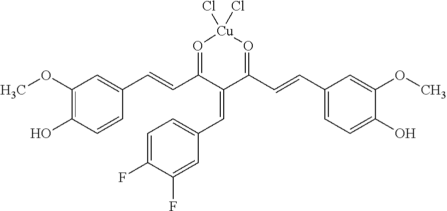

For the purposes of illustration, exemplary embodiments of Schiff Base ligands and copper conjugates that can be prepared from the compound of Formula II, and which fall within the scope of Formula I, include, but are not limited to:

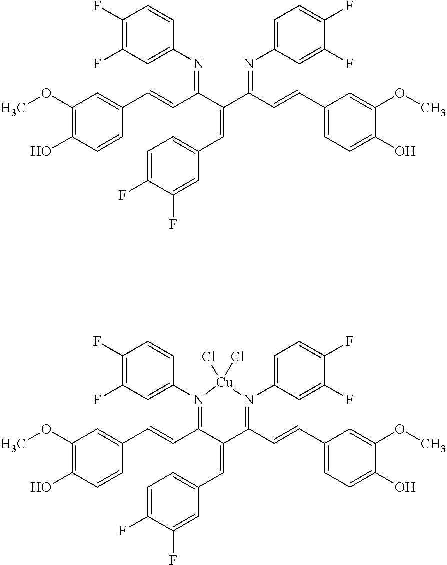

(1E,6E)-1,7-bis(4-hydroxy-3-methoxyphenyl)hepta-1,6-diene{4(3,4 difluorobenzaldehyde)}-3,5 bis (3,4 difluroro)aniline (CDFA; FIG. 1, Compound 6) and its copper conjugate (1E,6E)-1,7-bis(4-hydroxy-3-methoxyphenyl)hepta-1,6-diene{4(3,4 difluorobenzaldehyde)}-3,5 bis (3,4 difluroro)aniline Cu(II) (CDFACu; FIG. 1, Compound 11).

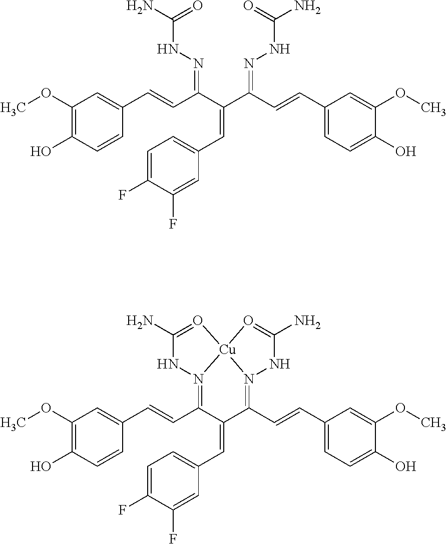

(1E,6E)-1,7-bis(4-hydroxy-3-methoxyphenyl)hepta-1,6-diene{4(3,4 difluorobenzaldehyde)}-3,5 bis hydrazine carboxamide (CDFS; FIG. 1, Compound 4) and its corresponding copper conjugate (1E,6E)-1,7-bis(4-hydroxy-3-methoxyphenyl)hepta-1,6-diene{4(3,4 difluorobenzaldehyde)}-3,5 bis(4-phenyl) hydrazine carboxamide Cu(II) (CDFSCu; FIG. 1, Compound 9).

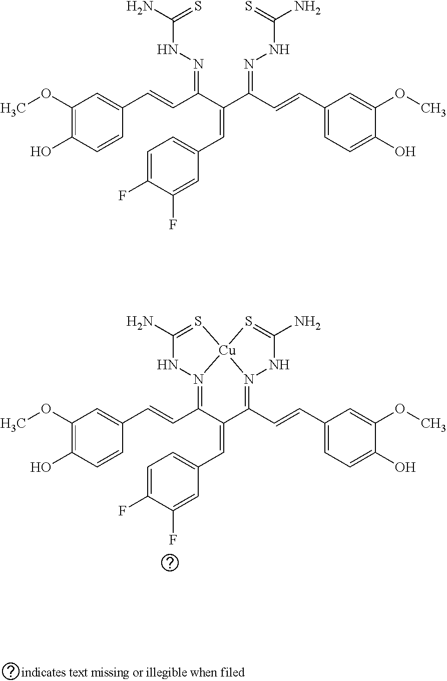

(1E,6E)-1,7-bis(4-hydroxy-3-methoxyphenyl)hepta-1,6-diene{4(3,4 difluorobenzaldehyde)}-3,5 bis hydrazine carbothioamide (CDFT; FIG. 1, Compound 5) and its copper conjugate (1E,6E)-1,7-bis(4-hydroxy-3-methoxyphenyl)hepta-1,6-diene{4(3,4 difluorobenzaldehyde)}-3,5 bis hydrazine carbothiomamide Cu(II) (CDFTCu; FIG. 1, Compound 10)

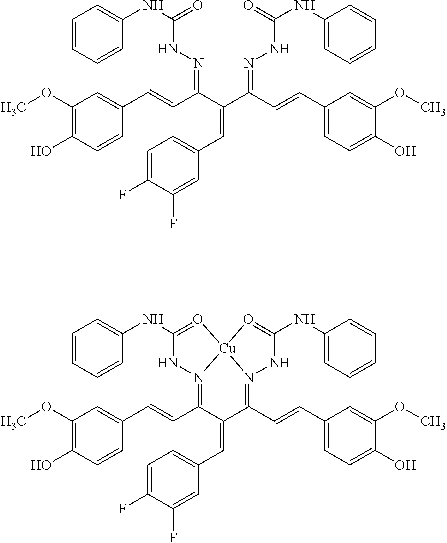

(1E,6E)-1,7-bis(4-hydroxy-3-methoxyphenyl)hepta-1,6-diene{4(3,4 difluorobenzaldehyde)}-3,5 bis(4-phenyl) hydrazine carboxamide (CDFI; FIG. 1, Compound 3) and its copper conjugate (1E,6E)-1,7-bis(4-hydroxy-3-methoxyphenyl)hepta-1,6-diene{4(3,4 difluorobenzaldehyde)}-3,5 bis hydrazine carboxamide Cu(II) (CDFI; FIG. 1, Compound 8).

While the compounds described herein have been directed to the particularly preferred copper conjugates, it is to be understood that other transition metals, such as nickel or platinum would be suitable in the practice of the invention. In this case, the ligand would be combined, in a stochiometric ratio, with a solution of a salt of the desired transition metal, which in specific illustrative embodiments, may be halides of copper, nickel, or platinum.

The molecules, once conjugated with copper, for example, have an E-tautomeric arrangement (square planar geometry). With respect to the geometrical isomers, the trans isomers are preferred.

In a further composition of matter aspect of the invention, a formulation comprises a therapeutically effective amount of a compound in accordance with the present invention in a delivery vehicle, or pharmaceutically-acceptable carrier. The compound may be the free drug or a pharmaceutically acceptable salt thereof. The term pharmaceutically acceptable salt includes, at least, the commonly used alkali metal salts used to form addition salts of free acids or free bases.

In a particularly preferred embodiment, formulation includes the difluorinated analog of Formula II, known as CDF, in a pharmaceutically acceptable carrier.

As used herein, the term “therapeutically-effective” refers to an amount of the compound that produces an ameliorating effect in the treatment and/or prevention of cancer, or other targeted disease, and is not toxic to the patient, and preferably does not produce excessive adverse side effects.

Clinically, it has been reported that a daily oral dose of 3.6 g of curcumin per day results in pharmacologically efficacious levels in colorectal tissue, with negligible distribution of the parent drug in hepatic, or other, tissues of the gastrointestinal tract. Krishnakutty, et al., Synth. Proact. Inorg. Met.-Org. Chem., Vol. 28, pages 1313-*(1998). It is also known that amounts of curcumin as great as 8 g per day does not result in toxicity. Therefore, it is expected that a person of skill in the art can devise a suitable dose for a patient in need of therapy.

It is contemplated that the compounds in accordance with the present invention can be formulated for delivery in any route of administration. For oral administration, the pharmacologic agent(s) can be delivered dry in the form of a tablet or capsule, or as a liquid solution or suspension. Oral drug delivery forms are well-known and typically include, conventional additives, such as binders and fillers, disintegrants, lubricants, and the like. For intravenous, intramuscular, subcutaneous, or intraperitoneal administration, the active pharmacologic agent may be combined with a sterile aqueous solution, such as saline or dextrose, preferably isotonic. Of course, a liquid injectable formulation can include other components, such as excipients, anti-oxidants, buffers, osmolarity adjusting agents, and the like, as are known in the art. In addition to conventional drug delivery approaches, it is within the contemplation of the invention that the pharmacologic agents of the present invention can be administered in targeted delivery media, such as in microparticle and nanoparticle formulations.

The compounds of the present invention can be used alone, or in combination, with other therapeutic agents, including anti-cancer agents, which may be known cytotoxic agents, such as genistein, celecoxib, gemcitabine, 5-flurouracil and oxaliplatin. The compounds can be used as an adjuvant, before, after, or concurrent with the administration of other medications or treatment therapies, such as radiation therapy.

In another aspect of the invention, there is provided a method of treating cancer and particularly pancreatic cancer or colon cancer, by administering a therapeutically effective amount of a compound of Formula I of Formula II in a pharmaceutically acceptable carrier either alone, or in combination with other known therapies, such as genistein, celecoxib, gemcitabine, 5-flurouracil and oxaliplatin.

In a specific preferred embodiment, of the invention, there is provided a method of inhibiting the growth of chemo-resistant colon cancer cells that are enriched in cancer stem-like cells in a subject having colon cancer comprising administering to the subject a therapeutically effective amount of a compound according to the invention in combination or conjunction with 5-FU and/or Oxaliplatin or FOLFOX.

In another specific preferred embodiment of the invention, there is provided a method of sensitizing pancreatic cancer cells and gemcitabine-resistant pancreatic cancer cells to gemcitabine for the prevention of tumor progression and/or treatment of pancreatic tumors in a subject comprising administering to the subject an effective amount of a pharmaceutical formulation comprising a compound in accordance with the invention, and particularly the compound of Formula II, in combination with gemcitabine.

### BRIEF DESCRIPTION OF THE DRAWING

Comprehension of the invention is facilitated by reading the following detailed description, in conjunction with the annexed drawing, in which:

FIG. 1 is a schematic representation of an illustrative synthetic procedure for preparing novel fluorinated analogs of curcumin in accordance with the present invention;

FIG. 2 is a graphical representation of the percent chymotrypsin-like activity (CT-like activity) assayed by measuring 20S proteasome inhibition in rabbit 20S proteasome at various concentrations of Curcumin (CUR) and the analogs CDF, CDFT, CDFS, CDFI, and CDFA;

FIG. 3 is a graphical representation of the percent CT-like activity assayed by measuring proteasome inhibition in human colon cancer HCT 116 cells at various concentrations of CUR and the analogs CDF, CDFT, CDFS, CDFI, and CDFA;

FIG. 4 is a graphical representation of the inhibition of cell growth (absorbance) of human colon cancer HCT 116 cells treated with various concentrations of CUR and the fluorine-substituted curcumin analogs CDF, CDFT, CDFS, CDFI, and CDFA;

FIG. 5 is a series of four bar graphs, labeled FIG. 5A through FIG. 5D showing the effect of CUR and the analog CDF, at various concentrations, on cell growth in human pancreatic cancer BxPC-3 (FIGS. 5A and 5B) and induction of apoptotic cell death (FIGS. 5C and 5D);

FIG. 6 shows in FIG. 6A an Electrophoretic Mobility Shift Assay for NF-κB DNA binding activity in MIA PaCa-2 cells exposed to CUR and CDF at the indicated concentrations; and FIG. 6B is a graphical representation of PGE₂ activity in conditioned medium derived from CDF- and CUR-treated BxPC-2 and MIA PaCa-2 pancreatic cancer cells;

FIG. 7 is a graphical representation of the concentration vs. time profile of CUR and CFD in mice serum (FIG. 7A) and pancreas tissue (FIG. 7B) following a single intragastric dose (250 mg/kg) of the stated compound to the mice;

FIG. 8 is a graphical representation of concentration vs. time profiling of curcumin (8A) and CDF (8B) in a variety of mouse tissues following single intragastric administration (250 mg/kg) of the compound in mice;

FIG. 9A to FIG. 9C are graphical representations of growth inhibition, in an MTT assay, of: BxPC-3 cells treated with 0.1 to 4.0 μMol/L of CDF and CUR (FIGS. 9A(1) and 9A(2)); BxPC-3 cells (FIG. 9B(1)), and gemcitibine-resistant cells, MIPaCa-E and MIPaCa-M (FIGS. 9B(2) and 9B(3), respectively) treated with 1-4 μMol/L CDF or CUR, 10 μMol/L gemcitibine (GEM), or a combination of GEM with CDF or CUR; and MIPaCa-E and MIPaCa-M (FIGS. 9C(1) and 9C(2), respectively) treated with 10 μMol/L CDF or CUR, 10 nMol/L GEM, or a combination of CDF/GEM or CUR/GEM. The p values represent comparisons between cells treated by either CDF or CUR, and their combination with GEM using the paired t test;

FIG. 9D is shows stained colony formation in the MIAPaCa-E cell line after being treated with 4 μMol/L CDF or CUR, 10 nMol/L GEM, and combinations thereof;

FIG. 10A are light photomicrographic pictures of BxPC-3, MIAPaCa-E and MIAPaCa-M cells showing change in morphology from epithelial-like to mesenchymal-like phenotype;

FIG. 10B is a series of three bar graphs (FIGS. 10B(1) to 10B(3)) showing the absorbance at 405 nm of the histone/DNA complexes produced in an ELISA assay of the three cell lines, BxPC-3, MIAPaCa-E, and MIAPaCa-M, respectively, following treatment with CUR, CDF, GEM, and the combinations CDF/GEM and CUR/GEM, at various concentrations;

FIG. 10C shows bar graphs of the absorbance at 405 nm of the histone/DNA complexes produced in an ELISA assay when the cell lines MIAPaCa-E and MIAPaCa-M were treated with a higher concentration of CDF and CUR (10 μMol/L) than in the FIG. 10B;

FIG. 11A comprises three Western blot chromatographs designating as FIG. 11A(1) to 11A(3), respectively, showing the expression of COX-2, E-cadherin, PTEN (phosphatase and Tensin Homolog), pAxt, tropomyosin, and β-actin in the cell lines BxPC-3, MIAPaCa-E, and MIAPaCa-M following treatment with CUR, CDF, GEM, and combinations of CDF/GEM and CUR/GEM at various concentrations;

FIGS. 11B(1) to 11B(4) are graphical representations of the comparative expression analysis of miR-21 in BxPC-3, MIAPaCa-E, and MIAPaCa-M by real-time miRNA reverse transcriptase-polymerase chain reaction (RT-PCR) following 72 hours of treatment with CUR, CDF, GEM, and combinations of CDF/GEM and CUR/GEM at various concentrations;

FIG. 11C is a chromatograph showing the expression of PTEN, pAκt, and NF-κβ in MIAPaCa-E cells after transfection with miR-21 antisense oligonuleotides;

FIG. 11D is a chromatograph showing the expression of PTEN, pAκt, and NF-κβ in MIAPaCa-E cells after transfection with PTEN cDNA;

FIG. 12A to 12B shows the EMSA assay for NF-κB DNA binding activity in BxPC-3, MIA PaCa-2, and MIAPaCa-E cells exposed to CDF, CUR, GEM, and combinations of CDF/GEM and CUR/GAM at the indicated concentrations;

FIG. 13A(1) to 13A(4) are graphical representations of the level of PGE₂ secretion determined by ELISA in BxPC-3, MIA PaCa-2, and MIAPaCa-E cell lines, respectively when treated with CDF, CUR, GEM, and combinations of CDF/GEM and CUR/GAM at the indicated concentrations;

FIGS. 13B(1) to 13B(3) are graphical representations of the level of VEGF secretion determined by ELISA in the treated BxPC-3, MIA PaCa-2, and MIAPaCa-E cell lines, respectively;

FIGS. 14A(1) and 14A(2) are graphical representations of the comparative expression of miR-200b miRNA and miR-200c miRNA in BxPC-3, MIA PaCa-2, and MIAPaCa-E cell lines as assessed by real-time RT-PCR;

FIG. 14B(1) to 14B(3) are graphical representations of the comparative expression of miR-200b miRNA in BxPC-3, MIA PaCa-2, and MIAPaCa-E cell lines, respectively, after treatment with CDF, CUR, GEM, and combinations of CDF/GEM and CUR/GAM at the indicated concentrations, as assessed by RT-PCT;

FIGS. 14C(1) to 14C(3) are the corresponding graphical representations for the comparative expression of miR-200c miRNA;

FIGS. 15A and 15B are graphical representations of the comparative expression of Lind28B miRNA and Nanog miRNA, respectively in AsPC-1, AsPC-1-GTR, MIAPaCa-2, and MIAPaCa-2-GTR cells as assessed by RT-PCT;

FIG. 15C is a Western blot chromatograph showing protein expression of EpCAM and CD44 in the cell lines represented in FIGS. 15A and 15B;

FIG. 16A shows stained colony formation in AsPC-1, AsPC1-GTR, MIAPaCa-2, and MIAPaCa-2-GTR cell lines after being treated with 4 μMol/L CDF or CUR, and 20 nMol/L GEM;

FIG. 16B is a series of four graphical representations of the fluorescence of the invaded AsPC-1, AsPC1-GTR, MIAPaCa-2, and MIAPaCa-2-GTR cell lines following treatment by CUR and CDF at 530/590 nm;

FIG. 16C is a chromatographic representation of ABCG2 expression in the AsPC-1, AsPC1-GTR, MIAPaCa-2, and MIAPaCa-2-GTR cell lines;

FIG. 17A to 17D are graphical representations of growth inhibition, in an MTT assay, of AsPC-1, AsPC1-GTR, MIAPaCa-2, and MIAPaCa-2-GTR cell lines, respectively, following treatment with CDF, CUR, GEM, and combinations thereof, along with a plot of the combination index;

FIGS. 18A to 18D are graphical representations of the disintegration of pancreatospheres/1000 cells as a result of treatment with CDF, CUR, and GEM in the AsPC-1, AsPC1-GTR, MIAPaCa-2, and MIAPaCa-2-GTR cell lines, respectively;

FIGS. 19A and 19B are graphical representations of the formation of pancreatospheres in AsPC-1 cells after 1 week (FIG. 19A) or 4 weeks (FIG. 19B) of treatment with CDF or CUR at 2.5 μMol/L, GEM at 20 nMol/L, or the CDF/GEM and CUR/GEM combinations;

FIGS. 19C(1) and 19C(2) are graphical representations of the formation of pancreatospheres/500 cells in AsPC-1 cells (FIG. 19C(1)) and AsPC-1 cells that have been pretreated in CDF (FIG. 19C(2)) in the presence of GEM and CDF in varying concentrations;

FIG. 20A is a graph of the anti-tumor activity of CDF, CUR, GEM, and the combination therapies, CDF/GEM and CUR/GEM, plotted as tumor weight (mg) against days, post-implant, of MIAPaCa-1 cell induced-tumors in mice;

FIG. 20B is an image of NF-κB DNA binding activity of tumor tissues and NF-κB competition control study with unlabeled NF-κB oligonucleotide;

FIG. 20C is a Western blot chromatograph showing COX-2, PTEN and β-actin expression in tumor remnants following treatment with CDF, CUR, GEM, and the combination therapies, CDF/GEM and CUR/GEM;

FIGS. 20D(1) to 20D(3) are graphical representations of miR-21, miR-200b and miR-200c expression, respectively, following treatment of tumor remnants with the stated agents, as measured by real-time RT-PCR;

FIG. 21A is a graphical representation of the growth of pancreatospheres in mice plotted as a function of tumor weight (mg) versus time post-inoculation;

FIG. 21B is a graphical representation of the comparative expression of miRNA in tumors derived from implanted MiPaCa-2 cells and from implanted pancreatospheres;

FIG. 21C are photographs showing tumor growth on a euthanized mouse. The arrow points to the main tumor and the asterisk (*) refers to loco-regional lymph node metastasis. No metastasis was found in the tumor derived from the parental cells; and

FIG. 21D is a graphical representation of the number of pancreatospheres in tumor cells harvested from the tumors derived from pancreatospheres which we untreated (control) or treated with CDF.

### DETAILED DESCRIPTION

### I. Synthesis of Curcumin Analogs

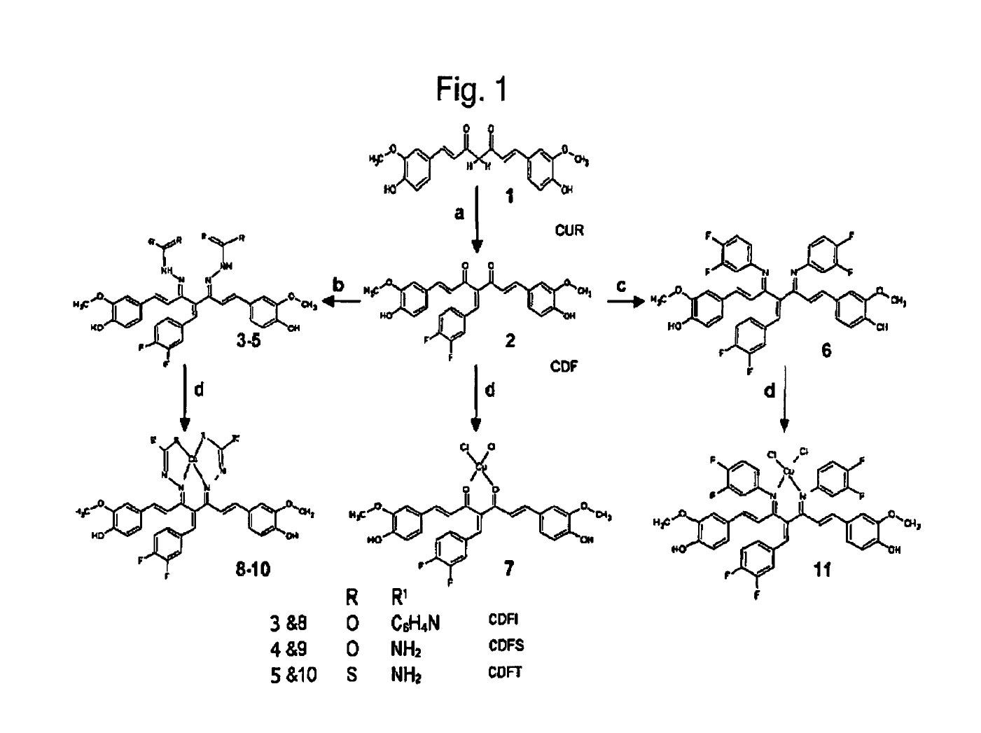

Specific illustrative methods of making curcumin analogs in accordance with the present invention are shown in Schema-1 in FIG. 1.

Curcumin, (1E,6E)-1,7-bis(4-hydroxy-3-methoxyphenyl)hepta-1,6-diene-3,5-dione, also known as diferuloylmethane, is shown on FIG. 1 as Compound 1. Curcumin Compound 1 was separated from a commercially purchased curcuminoid sample (Sigma-Aldrich, St. Louis) by column chromatography over silica gel using chloroform: methanol (9:1) as the eluting solvent. The purified curcumin was dissolved in a minimum amount of methanol to which an aldehyde, in methanol, was slowly added with stirring.

The purified curcumin is reacted with an aldehyde, in the presence of a weak basic amine catalyst, which in this case was piperidine. In the preferred embodiments shown in FIG. 1, the aldehyde is a fluoroaldehyde, and, specifically, 3,4 difluoroaldehyde. The reaction mixture was stirred for 48 hr and then set aside for product separation. The precipitated product was washed with adequate quantities of n-hexane and recrystallized from a chloroform-hexane mixture to yield pure dark brown microcrystalline product, Knoevenagel condensate (a 3,5 di-keto product) Compound 2.

In the specific embodiment shown in FIG. 1, Compound 2, coded as CDF, is (1E,6E)-1,7-bis(4-hydroxy-3-methoxyphenyl)hepta-1,6-diene{4(3,4 difluorobenzaldehyde)}-3,5-dione. (C₂₈H₂₂F₂O₆; Exact Mass: 492.14; M. Wt.: 492.47; m/z: 492 (100%), 493.14. (30.8%), 494.15 (4.6%), 494.14 (1.2%); Elemental Analysis: C, 68.29; H, 4.50; F, 7.72; O 19.49).

The condensates are reacted with different amines, or hydrazides to yield the corresponding bis-Schiff base ligands following a procedure described by Zambre, et al., Copper Conjugates of Knoevenagel Condensates of Curcumin and Their Schiff Base Derivatives: Synthesis, Spectroscopy, Magnetism, ESR, and Electrochemistry, Synthesis and Reactivity in Inorganic, Metal-Organic, and Nano-Metal Chemistry. 37:19-27 (2007).

Referring to FIG. 1, the condensate Compound 2 was dissolved in methanol and reacted with a hydrazide (1 mol condensate:2 mol hydrazide), in the presence of piperidine, with stirring for 24 hours at room temperature, and set aside for precipitation. In the specific embodiments shown on FIG. 1, the result was Compounds 3-5, herein coded as CDFI, CDFS, and CDFT, respectively.

In an alternative embodiment, the condensate CDF Compound 2 was reacted with an amine, specifically 3,4, difluoroamine in the example shown on FIG. 1, under the same conditions as the hydrazide compounds, to form Compound 6, herein coded as CDFA.

Compounds 3-6 are 3,5-disubstituted Schiff Base ligands that are analogs of CUR having a difluorinated substituent at the active methylene 4-position of the 1,5-diaryl pentadienone. Upon interaction with an aqueous solution of copper chloride, the bis-Schiff base ligands (1:1 mol) dissolved in methanol, in the presence of a catalytic amount of piperidine yielded the mono-ligand copper complexes shown in FIG. 1 as compounds 8-10 and 11.

The Knoevenagel condensate, Compound 2, yielded the copper complex Compound 7 upon reaction with copper chloride, under similar conditions.

The compositional analysis of the copper complexes, Compounds 7-11, indicate 1:1 metal-to-ligand stoichiometries for the Knoevenagel condensates and their Schiff bases. Conductivity measurements in DMSO solvent revealed a non-electrolyte nature.

¹H NMR spectra were recorded on an FT-NMR Varian Mercury 300 MHz instrument. The electronic absorption spectra were recorded on a Spectronic Genesys-2 spectrophotometer while IR spectra were recorded in KBr pellets on FTIR 3400 Shimadzu spectrophotometer. Magnetic susceptibility was measured at 300K on Faraday Balance having field strength of 7000 KG. Electron Paramagnetic Resonance (EPR) spectra were recorded as the polycrystalline sample on Varian X-band spectrophotometer using 1,1-diphenyl-2-picrylhydrazy (DPPH) as calibrant.

IR spectrum of Curcumin (Compound 1) in its stable enolizable form exhibits the carbonyl stretching frequency at 1620 cm⁻¹ and an intramolecularly hydrogen bonded hydroxyl absorption at 3379 cm⁻¹ whereas in the case of Knoevenagel condensates the carbonyl stretch appears at 1655-1633 cm⁻¹ while the hydroxyl absorption is found to be absent due to loss of enolizable hydrogen. In the Schiff bases of Knoevenagel condensates, the carbonyl frequency is replaced by strong absorptions at 1595-1600 cm⁻¹ ascribed to azomethine stretching frequency and an additional band at 860 cm⁻¹ due to the thiocarbonyl stretch (in case of the thiosemicarbazone ligand), respectively. Upon complexation with copper ions the carbonyl frequency exhibits an upward shift due to back coordination effects of oximino nitrogen donor indicating its involvement in metal complexation. The new bands observed in the spectra of the copper complexes in the regions 550-590 and 360-390 are ascribed to γ(-O) and γ(-Cl) stretching frequencies, respectively.

All ligands exhibited absorptions in the region 200-450 nm in their electronic spectra, as recorded in DMSO solvent, which are due to intra-ligand electronic transitions. An intense band was observed in the range 450-650 nm for the copper complex which is due to ligand-to-metal charge transfer, while the additional band observed in the region 650-850 nm is characteristic of Cu(II) (2B1g->2Eg) transition in square planar copper compounds. The magnetic moments of these complexes (1.69-1.82 BM) indicate their monomeric nature and support their planar geometries. The X-band EPR spectra of the copper compounds in DMSO glass are typical of axial symmetry with g_{∥}>g_{⊥}>2.0023, indicating the presence of unpaired electron in d_{x2-y2} ground state. The Distortion Factor Values in the range 110-120 are typical for planar complexes, while the higher values indicate extent of distortion.

### II. Experimental Methods/Data

In order to demonstrate usefulness of these compounds as chemopreventive and/or therapeutic agents, the following in vitro tests were conducted to demonstrate whether the curcumin analogs inhibit of proteasome and cell growth, as well as the induce apoptotic cell death.

### A. Proteasome Inhibition

1. Chymotrypsin-Like Activity

Inhibition of proteasome has been associated with the cell-killing activity of curcumin, as well as other proteasome inhibitors, The fluorine-substituted curcumin analogs of the present invention were tested to ascertain whether they could target chymotrypsin-like (CT-like) proteasome activity as compared to curcumin.

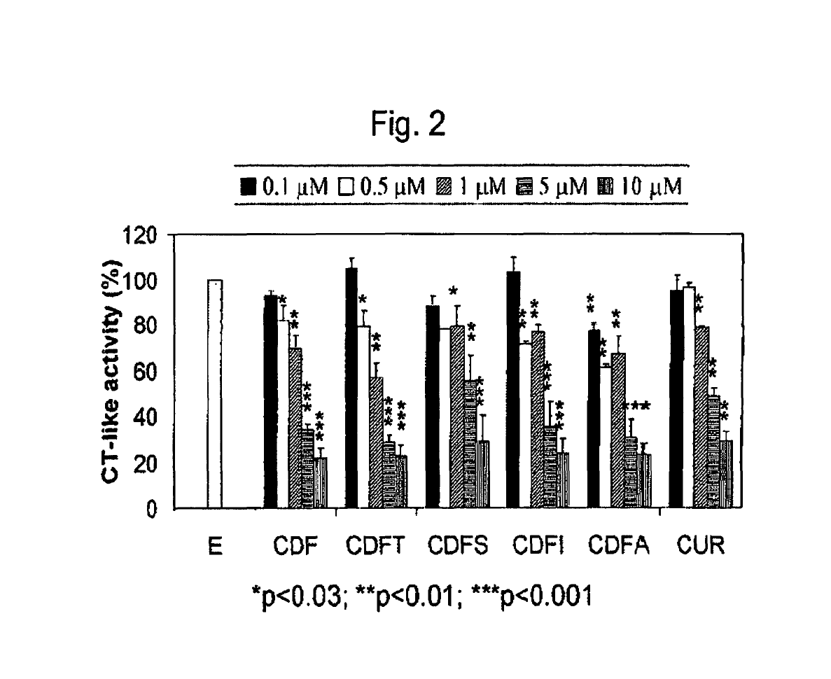

CT-like activity was assayed by measuring 20S proteasome inhibition by curcumin (CUR) and the fluorine-substituted curcumin analogs shown on FIG. 1 as Compound 2 (CDF), Compound 5 (CDFT), Compound 4 (CDFS), Compound 3 (CDFI), and Compound 6 (CDFA), at various concentrations: 0.1 μM, 0.5 μM, 1 μM, 5 μM, and 10 μM. Purified rabbit 20S proteasome and a fluorogenic substrate Suc-LLVY-AMC for the proteasomal chymotrypsin-like activity were obtained from Calbiochem Inc. (San Diego, Calif.). The purified rabbit 20S proteasome (35 ng) was incubated with 20 μM of the substrate Suc-LLVY-AMC in 100 μl assay buffer (20 mM Tris-HCl, pH 7.5, with curcumin or a fluorine-substituted curcumin analog at different concentrations, or in the solvent ethanol (E) for 2 hours at 37° C., followed by measurement of hydrolysis of the fluorogenic substrates using a Wallac Victor3™ multi-label counter with 355-nm excitation and 460-nm emission wavelengths. The results are shown on FIG. 2 which is a bar graph showing the % CT-like activity for the various concentrations of CUR and the fluorine-substituted curcumin analogs CDF, CDFT, CDFS, CDFI, CDFA.

Referring to FIG. 2, curcumin inhibited 20-70% chymotrypsin-like activity of the proteasome at 1-10 μM. At 0.5 μM, all fluorine-substituted curcumin analogs reached 20-40% proteasome inhibition. At high concentration, such as 10 μM, the fluorine-substituted curcumin analogs showed superior effects on proteasome inhibition as compared to curcumin (70-78% vs 70%).

In all of the examples herein, the statistical significance of the data was calculated using a paired two-tailed t-test following the GraphPad Prism software program and a p value of <0.05 was considered statistically significant when comparing treatment groups against control. Statistical significance is given on the figures.

2. Proteasome Inhibition in Human Colon Cancer HCT 116 Cells

Next, the fluorine-substituted curcumin analogs of the present invention were tested as to whether they could inhibit cellular 26S proteasome.

HCT 116 human colon cancer cells; human pancreatic carcinoma cell lines BxPC-3 and MIA PaCa-2, were purchased from American Type Culture Collection (Manassas, Va.) and grown in Dulbecco modified Eagle's medium supplemented with 10% fetal bovine serum (FBS), 100 units/ml of penicillin, and 100 μg/ml of streptomycin. Cells were maintained at 37° C. and 5% CO₂. FBS was obtained from Tissue Culture Biologicals (Tulare, Calif.). Penicillin and streptomycin were purchased from Invitrogen Co. (Carlsbad, Calif.).

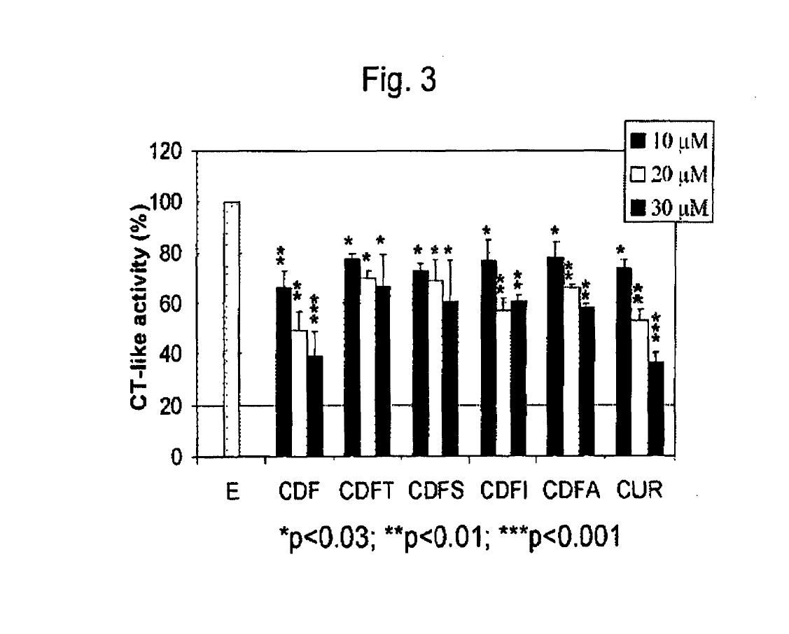

The human colon cancer HCT 116 cells were treated with various concentrations of CUR and its fluorine-substituted curcumin analogs for 24 hours, followed by proteasomal chymotrypsin-like activity assay. The results are shown on FIG. 3 which is a bar graph of proteasomal chymotrypsin-like activity (percent CT-like activity) assayed by measuring proteasome inhibition in human colon cancer HCT 116 cells at various concentrations of CUR and the fluorine-substituted analogs CDF, CDFT, CDFS, CDFI, CDFA. As a control, untreated cells were maintained in the solvent ethanol (E).

Referring to FIG. 3, the fluorine-substituted curcumin analog CDF exerted higher potency over the other analogs, showing 34%, 51%, and 61% proteasome inhibition at 10 μM, 20 μM, and 30 μM, respectively. This was similar to CUR which exhibited 27%, 47%, and 64% proteasome inhibition, respectively, at the same concentrations. The other fluorine substituted-curcumin analogs, specifically, CDFT, CDFS, CDFI, and CDFA, inhibited around 23-42% proteasome activity at 10-30 μM. These results suggest that CDF is biologically superior to curcumin.

3. Proteaseome Inhibition in Living Colon Cancer Cells by MTT Assay

To measure inhibition of the proteasome activity in living tumor cells by CUR and the fluorine-substituted analogs, about 5,000˜8,000 human colon cancer HCT 116 cells were planted in each well of a 96-well plate. Triplicate wells of cells were treated with CUR the or the analogs CDF, CDFT, CDFS, CDFI, and CDFA at different concentrations for 24 hours. The concentrations were 10 μM, 20 μM, and 30 μM. After aspiration of growth medium, (3-(4,5-Dimethylthiazol-2-yl)-2,5-diphenyltetrazolium bromide (MTT; 1 mg/ml) was added to the cell cultures, followed by incubation for 3 hours at 37° C. After the cells were crystallized, the MTT was removed and dimethylsulfoxide (DMSO) was added to dissolve the metabolized MTT product. The absorbance was then measured on a Wallac Victor³ 1420 Multi-label counter at 540 nm.

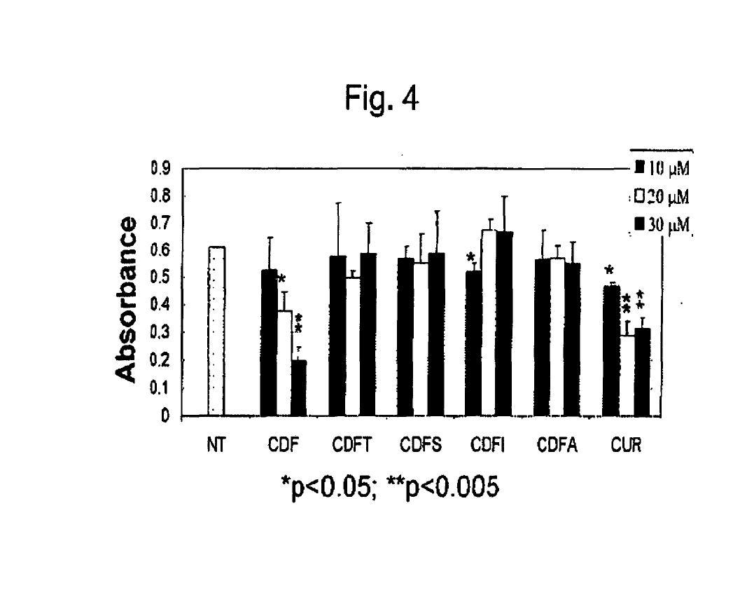

The results are shown in FIG. 4 which is a bar graph showing inhibition of cell growth of human colon cancer HCT 116 cells by the amount of color change in an MTT assay (absorbance) following treatment for 24 hours with various concentrations of CUR and the fluorine-substituted Curcumin analogs, or no treatment (NT). CDF was the most potent inhibitor showing 24%, 39%, and 68% inhibition on cell proliferation at 10 μM, 20 μM, and 30 μM, respectively, compared to curcumin with 24%, 53%, and 49% inhibition at the same respective concentrations.

4. Proteasome Inhibition in Human BxPC-3 PC Cells

(1) MTT Assay

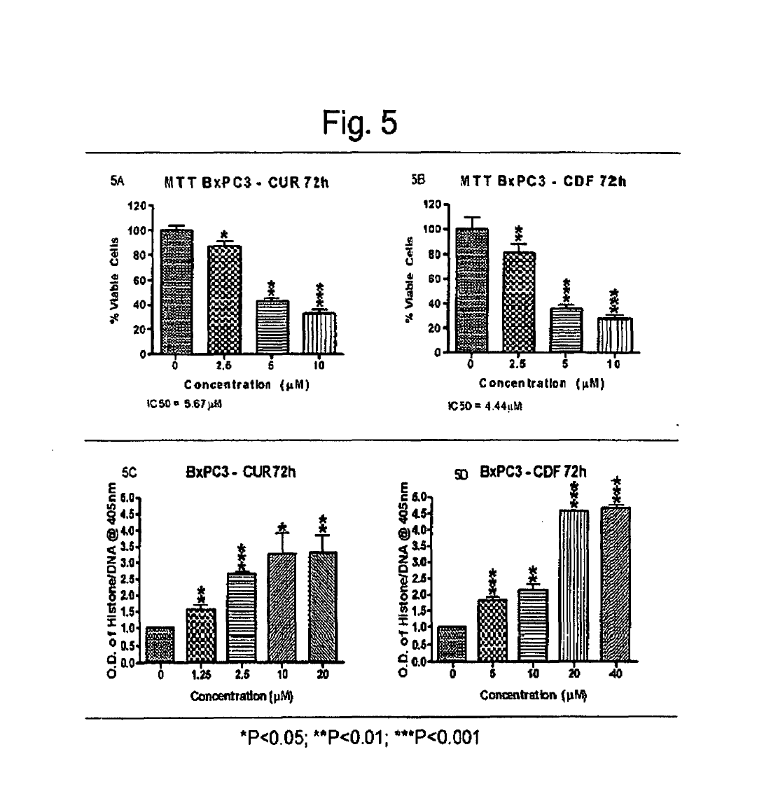

The inhibition of cell growth in BxPC-3 pancreatic cancer cells after 72 hours of treatment with CUR or the analog CDF was assessed by an MTT assay according to the procedure described above. The results are shown on FIG. 5 which is a series of four bar graphs, labeled FIG. 5A through FIG. 5D. FIGS. 5A and 5B show the effects of CUR and CDF on cell growth at various concentrations. CDF is superior to CUR in inducing cell growth inhibition.

(2) Cell Apoptosis Assay

A Cell Apoptosis ELISA Detection Kit (Roche, Palo Alto, Calif.) was used to determine whether the inhibition of cell growth could be due, at least partially, to the induction of apoptosis, in the BxPC-3 pancreatic cancer cells. BxPC-3 pancreatic cancer cells were treated with solutions of CUR or CDF, a various concentrations, for 72 hours. After the treatment, the cytoplasmic histone/DNA fragments were extracted and bound to immobilized anti-histone antibody. Subsequently, peroxidase-conjugated anti-DNA antibody was used for the detection of immobilized histone/DNA fragments. After addition of a substrate for peroxidase, the spectrophotometric absorbance of the samples was determined by using an ULTRA Multifunctional Microplate Reader (TECAN, Durham, N.C.) at 405 nM.

FIGS. 5C and 5D show the induction of apoptotic cell death in human BXPC-3 pancreatic cancer cells, after 72 hours of treatment with CUR and CDF, at various concentrations, in the cell apoptosis assay. Both CUR and CDF induce apoptosis. However, CDF is superior to CUR in this regard. These results are consistent with the MTT Assay results shown in FIGS. 5A and 5B.

The foregoing studies show that CDF is a potent analog of curcumin, which exceeds the potency of the parent in the inhibition of proteasome and cell growth, as well as the induction of cell death.

### B. Inactivation of NF-λB, and One its Downstream Targets, COX-2

The following experimental studies will demonstrate the superiority of CDF over CUR in the inactivation of NF-λB, and one its downstream targets, COX-2, through molecular modeling and corresponding bioassays.

1. Docking Studies

All calculations were performed using Auto Dock 3.05 software (Molecular Graphics, Laboratory, University of Georgia) The crystal structure of COX-2 protein was obtained from the RCSB Protein Database (PCD ID: 6COX). The active site of the enzyme was defined to include residues ALA 562, GLLU 346, GLN 350, within 0.65 radius, to any of the inhibitor atoms. Seven flurocurcumin analogs, as listed below on Table 1, and the parent CUR molecule were docked in the active site of the COX-2 enzyme

For each compound, the most stable docking model was selected based upon conformation of the best scored as predicted by the Auto Dock software scoring function. The compounds were energy minimized with an MMRR94 force field till the gradient convergence value of 0.05 kcal/mol was reached using distance dependence dielectric function ∈=4r.

The Results are shown on Table 1 which summarizes the docking results and consensus scores of flurocurcumin analogs, including CDF, and CUR.

TABLE 1
DOCKING RESULTS AND CONSENSUS SCORES
OF FLUROCURCUMIN ANALOGS

| | Docking | Binding | | | |
|---|---|---|---|---|---|
| | Energy | Energy | No. of 11 | II-bonding | |
| Molecules | (kcal/mol) | (kcal/mol) | Bonds | Residues | Log P |
| CUR | −7.78 | −5.71 | 1 | ALA 562 | 6.330 |
| CDF | −9.93 | −7.91 | 4 | GLU 346 | 4.321 |
| | | | | PIIE 580 | |
| | | | | ASN 101 | |
| | | | | GLN 350 | |
| CTF | −10.31 | −7.83 | 2 | GYL 354 | 4.280 |
| | | | | GLN 360 | |
| CTFM | −9.5 | −6.15 | — | — | 4.160 |
| CTETF | −9.27 | −7.36 | 2 | ALA 562 | 4.700 |
| | | | | GLN 192 | |
| CPF | −10.61 | −8.56 | — | — | 4.606 |
| CMF-1 | −6.84 | −2.61 | 2 | ASP 347 | 5.102 |
| | | | | AGN 109 | |
| CMF-2 | −9.63 | −7.17 | 2 | ALA 562 | 5.288 |
| | | | | GLO 346 | |

All flurocurcumin analogs tested were found to dock into the active site of COX-2, confirming that fluoro substitution does not introduce any major steric changes in the parent curcumin molecule. However, the flurocurcumin analogs allow more hydrogen bonding interactions. The binding energies of these analogs were in the range −2.6 to −8.56 kcal/mol compared to CUR's −5.71 kcal/mol. The lower interaction energy observed for CDF analog explains the tighter binding of this compound in the active site of COX-2 compared to the other analogs.

In the docking studies, CUR showed only one H-bonding interaction with ALA 562. On the other hand, the most potent fluoro analog, CDF, exhibits 4H-bonding interactions involving residues GLU 346, PHE 580, ASN101 and GLN 350. Other analogs (except CTFM and CPF) exhibited a maximum of two H-bonding interactions, the residue ALA 562 being in common with the parent curcumin. Favorable van der Waals interactions between styryl carbon atoms and hydrophobic residues, such as GLU 346 (3.01A), SER 353 (3.13A), or between methoxy group of CDF and HIS 351, ALA 582, contributed to stabilize the ligand-enzyme complexes. The lower liposolubility observed for the CDF analog suggests that CDF should have slower metabolism, with an enhanced pharmacokinetic profile, than the naturally-occurring parent CUR, which was confirmed by the studies as presented below.

2. NF-κB DNA Binding Activity in Pancreatic Cancer Cells

Since CDF docks into the active site of COX-2, which is transcriptionally regulated by NF-κB, an investigation was conducted to ascertain whether CDF would also have an effect on the nuclear transcription factor NF-κB, as well as COX-2 activity, in a manner similar to CUR. This was confirmed by measuring the effects of CDF on NF-κB DNA binding activity in MIAPaCa-2 cells, and PGE₂ production in both MIAPaCa-2 and BxPC-3 pancreatic cancer cells in the studies reported below.

(1) EMSA for NF-κB DNA Binding Activity in Pancreatic Cancer Cells

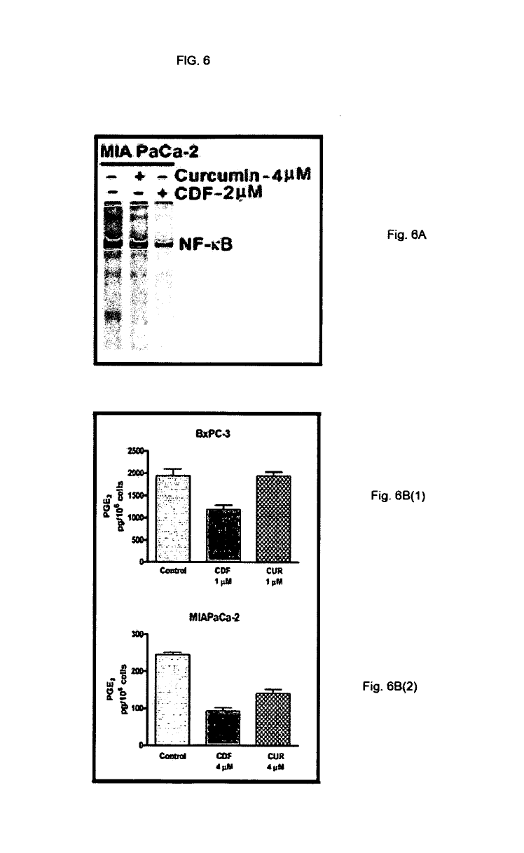

Nuclear extracts were prepared from treated samples of pancreatic cancer cells (MIA PaCa-2) that had been exposed to CUR and CDF in concentrations of 4 μM and 2 μM, respectively. An Electrophoretic Mobility Shift Assay (EMSA) was performed by incubating 10 μg of nuclear extract with IRDye™-700 labeled NF-κB oligonucleotide in a technique described more completely in Banerjee, et al., Cancer Res., Vol. 69, pages 5592-5600 (2009). The DNA-protein complex was visualized by an Infrared Imaging system and shown in FIG. 6A.

(2) PGE₂ Immunoassay for Quantitation of Prostaglandin E2F

BxPC-3 and MIA PaCa-2 cells were either untreated (control), or treated with CDF and CUR at 1 μM (FIG. 6B(1)) or at 4 μM (FIG. 6B(2)) for 24 hours. The conditioned medium was collected and analyzed for PGE₂ concentration, according to the manufacture's protocol, using a PGE₂ high sensitivity immunoassay kit purchased from R & D Systems, Minneapolis, Minn. The optical density was measured at 450 nm and the concentration of PGE₂ was calculated from the standard curve. The results, expressed as PGE₂ in pg/10⁶ cells, are shown on FIG. 6B.

As widely reported, CUR caused down-regulation of NF-κB, but the effect was even more pronounced at a low concentration of CDF. Additionally, both CUR and CDF caused a significant decrease in PGE₂ levels in MIAPaCa-2 cells as shown in FIG. 6B(2). However, in BxPC-3 cells, only the CDF-treated cells showed a significant decrease in PGE₂ level. These results clearly demonstrate that CDF is a better target of COX-2 than CUR, resulting in greater inhibition of PGE₂ production, in both the cell lines, as compared to the naturally-occurring curcumin.

### C. Pharmacokinetics and Tissue Distribution Studies

The pharmacokinetics and tissue distribution of CUR and CDF were examined in female 7-8 week old ICR-SCID mice purchased from Taconic Farms (Germantown, N.Y.). The mice were randomly divided into two groups of 18 mice apiece. One group was given a single-dose of CUR (250 mg/kg) diluted in 0.1 ml volume of sesame oil by intragastric intubation, and the second received a similar single-dose containing CDF (250 mg/kg). Blood and tissue samples were harvested before initiation of treatment (0 hr) and at 1, 2, 4, 6, 8, 12, 16, and 24 hrs following the intragastric administration. At each time point, two mice were euthanized and ˜200 μL blood was collected by cardiac puncture, and tissues (liver, lung, kidney, heart, pancreas, and colon) were harvested, washed free of blood with PBS, blotted dry, weighed, and stored at −80° C. until analysis. The collected blood samples were allowed to clot, centrifuged, and then the serum was separated and stored at −80° C. until analysis.

The CUR and CDF concentration in mouse serum and tissue samples were determined using validated high-performance liquid chromatography (HPLC) with tandem mass spectroscopy (LC-MS/MS). The tissue samples were homogenized in 5 volumes of ice-cold normal saline. Aliquots of serum or tissue homogenate were spiked with ethyl acetate (containing 50 ng/ml zilueton as an internal standard) as follows: 100 μL serum/500 μL ethyl acetate and 500 μL tissue homogenate/1 ml ethyl acetate. The mixtures were vortex-mixed and centrifuged at 14,000 rpm for 5 minutes. The top layer was separated and dried under a stream of nitrogen in a water bath at about 50° C. The residue was reconstituted in 100 μL of methanol/water containing 0.45% formic acid (70:30 v/v) and then centrifuged again.

The supernatant (100 μL) was injected into the HPLC equipment, and separated on a Waters Xterra MS column (2.1×50 mm, 3.5 μM i.d.) With a mobile phase consisting of methanol/water containing 0.45% formic acid (70:30 v/v) at a flow rate of 0.2 ml/min. The column effluent was monitored using a Waters Quattro Micro™ triple quadrapole mass-spectrometric detector equipped with an electrospray ionization source. (Waters Corporation, Milford, Mass.). CUR and CDF were monitored in the negative ionization mode at the transition of m/z, 367.1→148.8 and 491.1→216.9, respectively. The internal standard zilueton was monitored in the positive mode at the transition of m/z, 237.1→160.8. The calibration curves for CUR and CDF were constructed over the concentration range of 5 to 2,000 and 5 to 10,000 ng/ml, respectively for the serum and tissue samples. The within-day and between-day precision and accuracies of the assay was <15%.

Serum pharmacokinetic parameters were estimated using non-compartmental analysis with WinNonlin software version 4.2 (Pharsight Corporation, Cary, N.C.). The maximum serum concentration (Cₘₐₓ) and the time of occurrence for maximum concentration (Tₘₐₓ) were obtained by visual inspection of the serum concentration-time curve after drug administration. The total area under the serum concentration-time curve from time zero to the last measurable time point (AUCₘₐₓ) was calculated using the linear and logarithmic trapezoidal method for ascending and descending serum concentrations, respectively.

1. Serum and Tissue Data

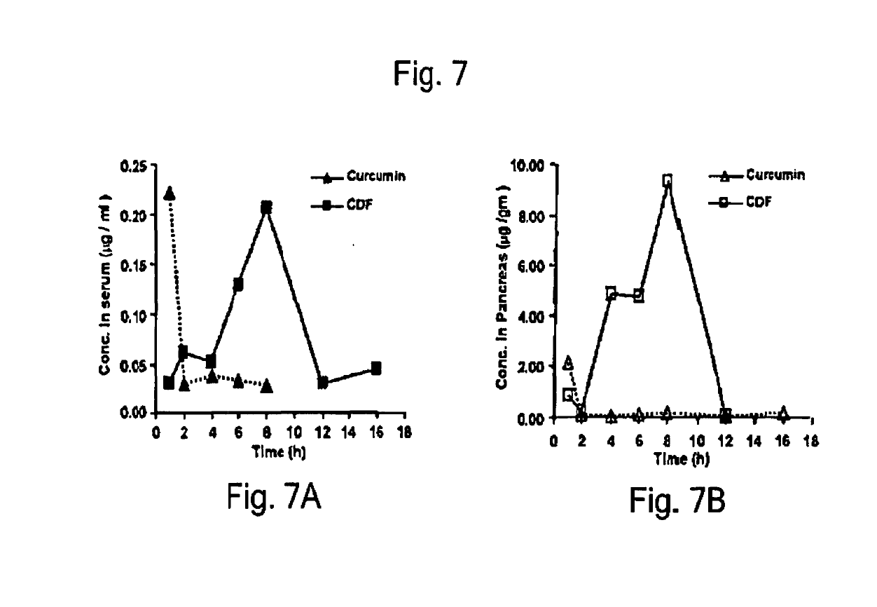

The concentration and time profiles of CUR and CDF in serum and pancreas tissue following a single dose oral administration (250 mg/kg) in female ICR-SCID mice are shown in FIGS. 7A and 7B, each point representing the mean concentration obtained from two mice.

Consistent with previous findings relating to low bioavailability, very low serum levels of CUR were observed after oral administration to mice. Following a single oral dose of 250 mg/kg, CUR achieved the Cₘₐₓ of 0.22 μg/mL at 1 h, after which CUR serum concentration declined rapidly and was undetectable after 8 h (below the lower limit of 5 ng/mL; see, FIG. 7A).

These results are consistent with a previously reported mouse study in which oral administration of 1 g/kg body wt. of curcumin resulted in a Cₘₐₓ of 0.22 μg/mL at 1 h, and the serum concentrations then declined below the detection limit by 6 h. See, Pan, et al., Biotransformation of curcumin through reduction and glucuronidation in mice, Drug Metab. Dispos., Vol. 27, pages 486-494 (1999). This has been attributed, by the prior art, to poor water solubility and extensive first-pass intestinal and hepatic metabolism, and contributes, in large measure, to the low oral bioavailability of curcumin. In contrast, the difluorinated analog CDF, of the present invention, exhibited enhanced bioavailability. Oral administration of CDF produced a 2.7-fold increase in systemic drug level over Curcumin (AUCₗₐₛₜ, 1.22 vs. 0.44 μg/mL at 1 h; See, Table 2 below).

2. Pharmcokinetic Data

The pharmacokinetic parameters for CUR and CDF are summarized in Table 2.

TABLE 2
Comparative pharmacokinetic analysis of Curcumin and CDF in
serum and pancreas following a single intragastric administration
(250 mg/kg) in mice. Data are expressed as the mean from two mice.

| | Serum | | Pancreas | |
|---|---|---|---|---|
| | Curcumin | CDF | Curcumin | CDF |
| Tₘₐₓ (h) | 1.0 | 8.0 | 1.0 | 8.0 |
| Cₘₐₓ (μg/mL for serum μg/g | 0.22 | 0.21 | 2.15 | 9.35 |
| for pancreas) | | | | |
| Tₗₐₛₜ (h) | 8.0 | 16.0 | 16.0 | 12.0 |
| Cₗₐₛₜ (μg/mL for serum μg/g | 0.03 | 0.04 | 0.20 | 0.04 |
| for pancreas) | | | | |
| AUCₗₐₛₜ (h * μg/mL for serum h * | 0.44 | 1.22 | 3.46 | 36.56 |
| μg/g for pancreas) | | | | |

Abbreviations:
Cₘₐₓ, maximum serum concentration
Tₘₐₓ, the time to achieve maximum concentration
Cₗₐₛₜ, last measurable concentration
Tₗₐₛₜ, the time for the last measurable concentration
AUCₗₐₛₜ , total area under the serum concentration-time curve from time zero to the last measurable time point

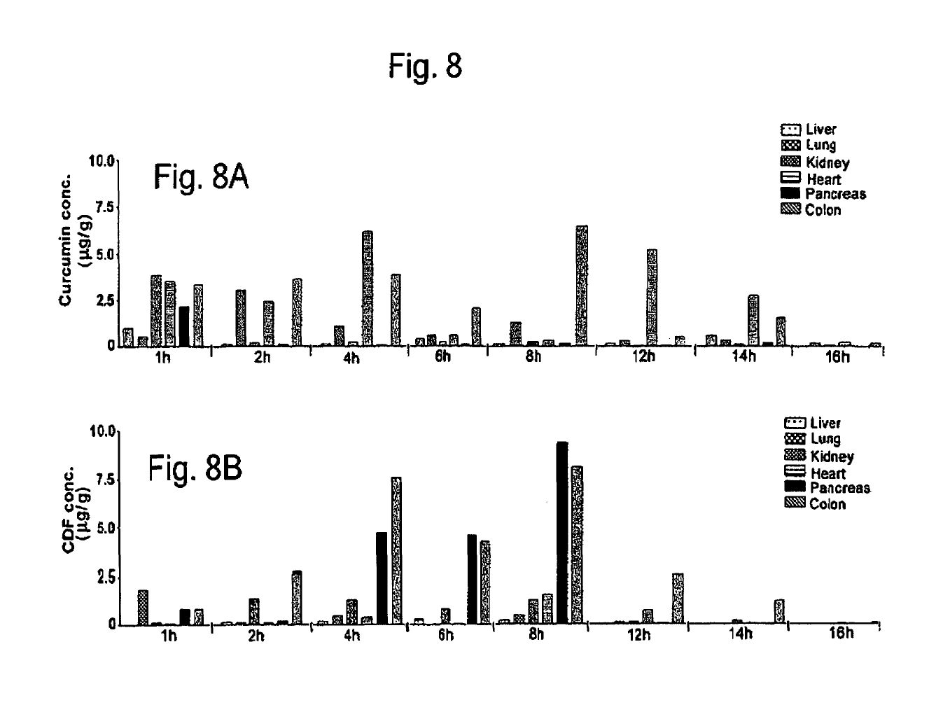

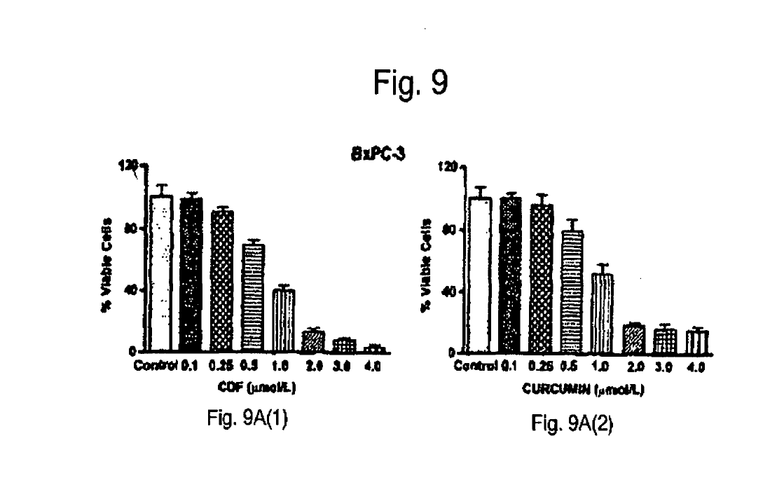

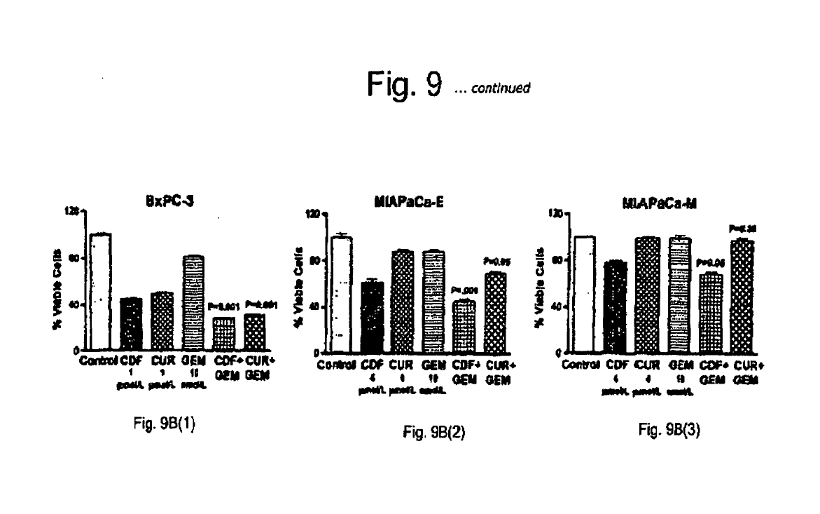

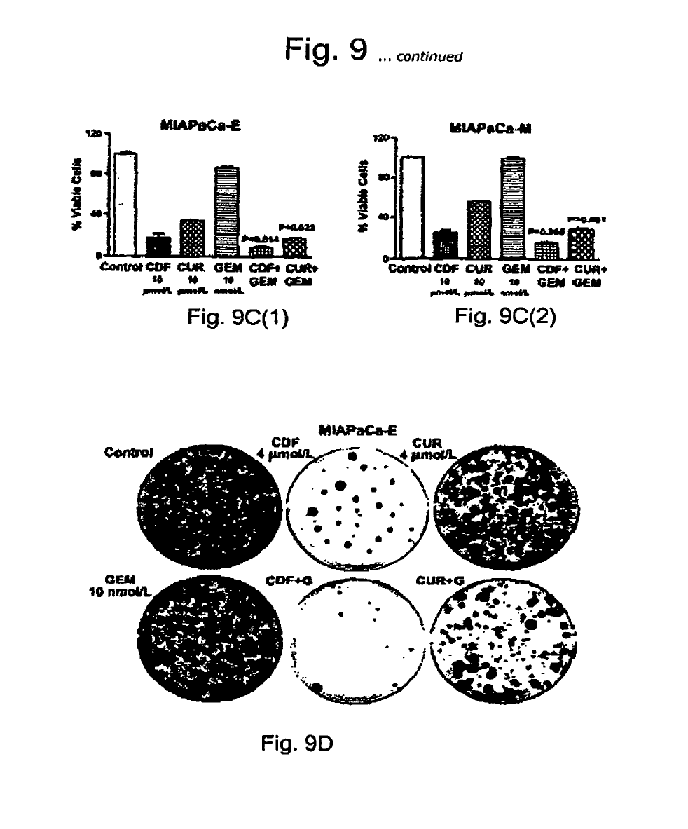

The distribution of CUR and CDF following single dose administration of 250 mg/kg body weight in mice is presented in FIGS. 8A and 8B, respectively. As shown in the FIGS. 8A and 9B, both CUR and CDF were detectable in all tissues tested, including liver, lung, kidney, heart, pancreas, and colon. Both CUR and CDF were detectable at high concentrations in the colon after oral administration. Interestingly, CUR was found to be present mainly in heart and lung tissue, while CDF accumulated preferentially in the pancreas (See, FIGS. 8B and 9B). The Cₘₐₓ (at 8 h) of CDF achieved in pancreas was 44.5 fold higher than in serum (FIGS. 7 and 8; Table 2). Consistent with the serum concentration vs. time profile shown in FIG. 7B, CUR and CDF achieved the maximum concentration in the pancreas at 1 and 8 h, respectively, after oral administration. The Cₘₐₓ and AUCₗₐₛₜ of CDF in pancreas tissue were 4.3 and 10.6 times that of those for CUR (See, Table 2), suggesting that CDF has the better bioavailability profile, especially in pancreatic tissue. Therefore, demonstrating the usefulness of CDF for anti-tumor activity against pancreatic cancer.

The high accumulation of CDF in the pancreas makes CDF a good candidate for inclusion in a prevention strategy for pancreatic cancer and/or for the treatment of pancreatic cancer, either alone or in conjunction with other cytotoxic agents, such as the current chemotherapeutic agent of choice, gemcitabine.

### D. Use of CDF for the Treatment of Drug-Resistant Cancers

The studies conducted in this Section demonstrate that CDF up-regulates miRNA-200b and miRNA-200c, and down regulates miR-21 in both gemcitabine-sensitive (BxPC-3), and gemcitabine-resistant (MIAPaCa-E and MIAPaCa-M) cell lines. This action has been associated with induction of apoptosis.

1. Creation of GEM-Resistant Pancreatic Cancer Cell Lines

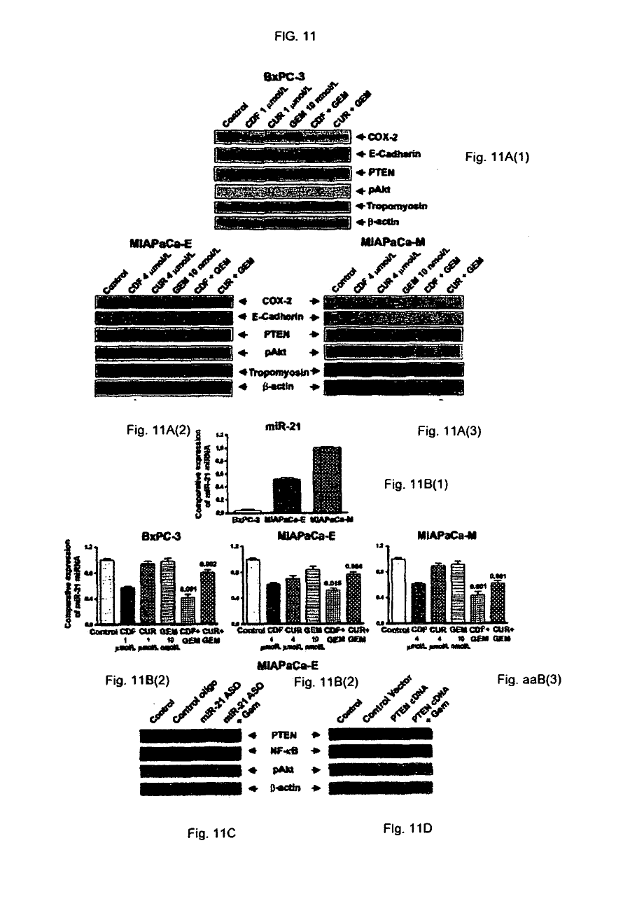

The human pancreatic cancer cell lines MIAPaCa-E, MIAPaCa-M, and BxPC-3 were chosen for this study based on their sensitivity to gemcitabine. The MIAPaCa cells were exposed to gemcitabine every other week for four months to create a gemcitabine-resistant cell line that could be compared to (paired) non-resistant cell lines, which resistant cell lines are identified herein as MIAPaCa-E and MIAPaCa-M, based on the changes in morphology from epithelial-like to mesenchymal-like phenotype as shown in FIG. 11A which are light photomicrographs of the cell lines. The MIAPaCa-E cell line is relatively resistant to GEM, however, the MIAPaCa-M is highly resistant to GEM. The cell lines have been tested and authenticated by the Applied Genomics Technology Center at Wayne State University, Detroit, Mich., using short tandem repeat (STR) profiling with the PowerPlex® 16 System from Promega (Madison, Wis.).

(1) MTT Assay

Cell growth was assessed by an MTT assay in a manner similar to that as described above. The color intensity was measured by a TECAN microplate fluorometer (Tecan Group, LTD, Research Triangle Park, N.C.) at 595 nm.

To test cell viability, 3,000 cells/well were plated in a 96-well plate and stored for 24 h. Initially, the cells were subjected to a range of concentrations of CDF and CUR (0.1-4.0 μMol/L) and GEM (10-50 nMol/L). Based on the initial results for BxPC-3 cells, a concentration of 1 μMol/L of CDF or CUR and 10 nMol/L of GEM (10 nMol/L) was used in all subsequent assays. Higher doses of CDF and CUR (4 or 10 μMol/L) and GEM (10 nMol/L), and their combinations, were used to test the effects of treatment on the GEM-resistant cell lines.

Referring to FIGS. 9A(1) and 9A(2), significant reduction in cell viability was seen in BxPC-3 cells treated with CDF and CUR. Based on the results shown in FIGS. 9A(1) and 9A(2), a concentration of 1 μMol/L of CDF and CUR and 10 nMol/L of GEM was used in the combination experiments on BxPC-3 as shown FIG. 9B(1), which also indicates significant inhibition of BxPC-3 cell viability in the combination treatments by CDF/GEM and CUR/GEM. The results for the GEM-resistant cell lines MIAPaCa-E and MIAPaCa-M cells are shown in FIGS. 9B(2) and 9B(3)

The GEM-resistant cell lines MIAPaCa-E and MIAPaCa-M cells were also treated with higher concentrations of CDF and CUR (10 μMol/L) and GEM (10 nMol/L). As shown in FIGS. 9B(2) and 9B(3) and FIGS. 9C(1) and 9C(2), the combination of CDF/GEM and CUR/GEM showed greater inhibition than CDF or CUR alone. The combination CDF/GEM was the most effective in all cell lines.

(2) Clongenic Assay

The effect of treatment on cell growth was also assessed by clonogenic assay. MIAPaCa-E cells were plated (50,000 cells/well) in a six well plate. After 72 h exposure to 4 μMol/L of CDF or CUR, 10 nMol/L of GEM, or the combination CDF/GEM or CUR/GEM, the cells were trypsinized. Then, 1000 viable cells were plated in 100 mm petri dishes. The plated cells were then incubated for about 10-12 days at 37° C. in an incubator under an atmosphere of 5% CO₂/5% O₂/90% N₂. The cell colonies were stained with 2% crystal violet and quantitated. Images are shown on FIG. 9D.

Referring to FIG. 9D, the CUR/GEM and CDF/GEM combination treatments resulted in a significant inhibition of colony formation in MIAPaCa-E cells as compared to single agent treatment. The effect was more pronounced for CDF/GEM than for CUR/GEM. Similar results were observed in the other cell lines (data not shown). Based on the differential sensitivities of these cell lines to the above mentioned agents, all subsequent experiments were conducted using CDF/CUR concentrations of 1 μMol/L for the BxPC-3 cell line and of 4 μMol/L for the MIAPaCa-E and MIAPaCa-M cell lines.

(2) Cell Apoptosis Assay

The underlying mechanism on the inhibition of cell viability was further studied by determining the apoptotic effects using the Cell Death Detection ELISA kit (Roche Applied Science, Indianapolis, Ind.) in a manner similar to that disclosed above in Section A(4)(2), and as described in Ali, et al., Mol. Cancer Ther., 4:1943-1951 (2005).

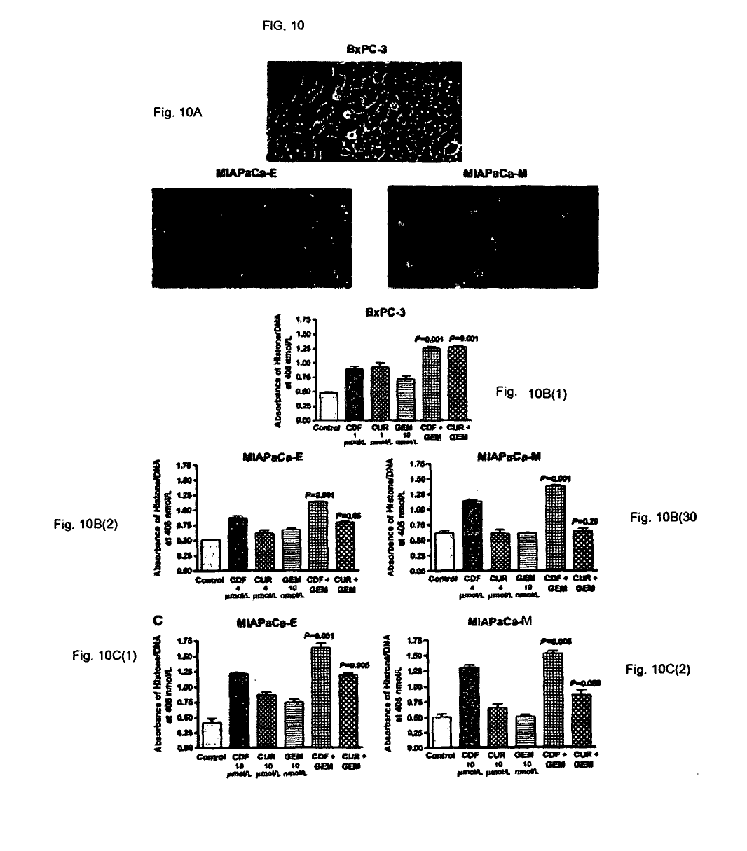

In this study, BxPC-3, MiPaCa-E and MIAPaCa-M cells were treated with 1 to 4 μMol/L of CDF or CUR, and 10 nMol/L of GEM, or the combinations of CDF/GEM or CUR/GEM. The results are shown in the graphical representations on FIG. 10B(1) to FIG. 10B(3). The combination CDF/GEM resulted in significant induction of apoptosis in all three cell lines, and greatly exceeded induction of apoptosis in the MIAPaCa-E and MIAPaCA-M cell lines as compared to CUR/GEM, or any of the agents alone. Increasing the concentration of CDF and CUR in the treatment solution from 4 μMol/L to 10 μMol/L for the MIAPaCa-E and MIAPaCa-M cell lines, resulted in even greater induction of apoptosis in cells treated with both CDF/GEM and CUR/GEM as shown in FIGS. 10C(1) and 10C(2). These results are consistent with the cell viability assay reported hereinabove.

### E. Treatment Effects on the Expression of Selective Proteins

The following studies were conducted to investigate the mechanism(s) behind the differing effects of CDF, CUR, GEM, and the combinations thereof, on the three cell lines BxPC-3, MiaPaCa-E and MiaPaCa-M.

1. Western Blot: COX-2, E-Cadherin, PTEN, pAkt, Tropomyosin, and β-Actin

Cells were treated with CDF or CUR (1 or 4 μMol/L), GEM (10 nMol/L), and the combinations of CDF/GEM and CUR/GEM for 72 h. The results of the Western blot analysis for the three cell lines are shown in FIG. 11A(1) to 11A(3). There was a complete loss of basal level expression of COX-2 in the MIAPaCa-M cells, which were prepared by exposure to GEM for a period of about four months and are the most resistant to the effects of GEM. Expression of COX-2, pAκt and tropomyosin proteins was significantly reduced in cells treated with CDF/GEM as compared to CUR/GEM. Epithelial marker E-cadherin expression was significantly enhanced by treatment with CDF, CUR, and the combinations of same with GEM, only in the BxPC-3 cell line. No such effect was observed in the GEM-resistant MIAPaCa-E and MIAPaCa-M cell lines due to lack of a basal level of expression of E-cadherin, which is consistent with the EMT phenotype. The expression of PTEN, a tumor suppressor gene, was decreased in the MiaPaCa-M cell line as compared to the BxPC-3 cell line, and was up-regulated with the treatment combinations. These results suggest that CDF is much more effective than CUR.

Since PTEN is a known target of miR-21, which has been reported to be up-regulated in pancreatic cancer, the following study was conducted to assess the expression levels of miR-21 and its interrelationship with the expression of PTEN in PC cells.

2. PTEN cDNA Transfection: PTEN, pAkt, NF-κB, and β-actin

To determine the effect of PTEN cDNA transfection, MIAPaCa-E cells were plated in 100 mm petri dishes overnight, transfected with either 15 μg of PTEN cDNA or a control empty vector by ExGen 500 (Fermentas, Hanover, Md.) following the manufacture's protocol. Cells were treated with GEM (10 nMol/L), or left untreated, for 48 h and assessed for the expression of PTEN, pAkt, NF-κB, and β-actin by Western blot analysis.

The results are shown in FIG. 11B(1) to FIG. 11B(4) which are graphical representations of the comparative expression analysis of MiR-21 in the BxPC-3, MIAPaCa-E, and MIAPaCa-M cell lines by real-time miRNA PCR when treated with CUR, CDF, GEM, CUR/GEM and CDF/GEM at the specified concentrations. Over-expression of miR-21, which is an oncogenic miRNA that has shown anti-apoptotic activity in various carcinomas cell lines, was observed in both GEM-resistant cell lines, MIAPaCa-E and MIAPaCa-M, as compared to BxPC-3 cells.

Referring to FIG. 11B, there was a significant reduction in the expression of miR-21 in cells treated with either CDF or the combination CDF/GEM. To validate further that miR-21 targets the expression levels of the tumor suppressor PTEN, an experiment was conducted to study the effect of transfection of PTEN cDNA and miR-21 antisense oligonucleotide on MIAPaCa-E cells.

3. Antisense miR Oligonuleotide Transfection: PTEN, pAkt, and NF-κB

Because the MIAPaCa-E cell line showed increased expression of miR-21, this cell line was used to evaluate the effect of both PTEN cDNA and miR-21 antisense oligonucleotide transfection on the expression of PTEN, pAkt, and NF-B. The cells were transfected with PTEN cDNA, miR-21 antisense oligonucleotide, or a nonspecific control vector (ExGen 500, Fermentas, Hanover, Md.), for 48 h, and then treated with GEM for 48 h. The transfection efficiency of the targeted proteins PTEN, pAkt, NF-κB, and β-actin was assessed by Western blot analysis. The resulting chromatographs are shown in FIG. 11C and FIG. 11D.

The expression of PTEN was enhanced by both transfection studies compared to either untreated cells or cells treated with control vector or oligos, respectively. On the other hand, the expression of pAκt and NF-κβ was further reduced with both the transfections (see, FIGS. 11C and 11D). These findings clearly suggest that CDF and CUR are capable of re-activating the expression of PTEN, which is normally lost in malignant tumors, and that the reactivation is mediated by down-regulating the expression of miR-21.

In order to gain insight on the mechanism by which CDF and CUR sensitizes PC cell lines to GEM-induced inhibition of cell viability and induction of apoptosis, the DNA binding activity of NF-κβ was assessed by the effects of the various treatments on NF-κβ downstream genes, such as COX-2.

4. EMSA for NF-κB DNA Binding Activity

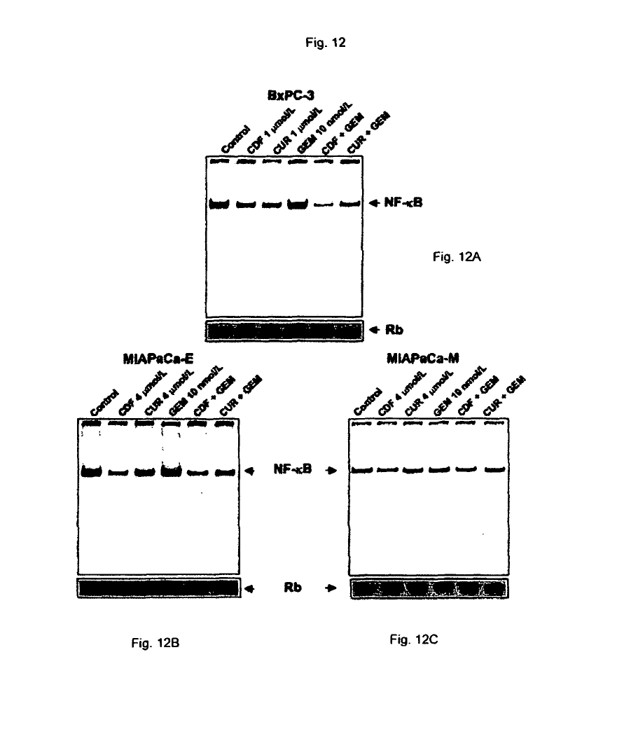

The DNA binding activity of the nuclear transcription factor, NF-κB was assessed in the BxPC-3, MIAPaCa-E, and MIAPaCa-M cell lines by EMSA. The cells were either untreated or treated with CDF/CUR (1 or 4 μMol/L), GEM (10 nMol/L), or a combination of CDF/GEM and CDF/CUR, for 72 hours. The cells were lysed in 400 μl of lysis buffer and the reaction was set up as described earlier in Ali, et al., id. The gel was scanned using an Odyssey Infrared Imaging System, (LI-COR, Inc., Lincoln, Nebr.). Equal protein loading was ensured by immunoblotting 10 μg of nuclear protein and probing with anti-retinoblastoma antibody. The results are shown in FIG. 12.

Referring to FIG. 12, GEM treated cells caused activation of NF-κβ in all three cell lines, however, CDF treated cells caused significant inhibition in the DNA binding activity of NF-κβ in all three cell lines. Interestingly, GEM-induced activation of NF-κB was attenuated by CDF treatment. However, the cells treated by CUR, or the CUR/GEM combination, showed much lower effects on NF-κβ DNA binding activity than the CDF-treated cells. This suggests that the combination of CDF/GEM causes greater inhibition of cell growth, better induction of apoptosis, and greater inhibition of COX-2 protein; all of which could be, in part, due to inactivation of NF-κβ in both gemcitabine-resistant and gemcitabine-sensitive cell lines.

5. Determination of PGE₂ Levels

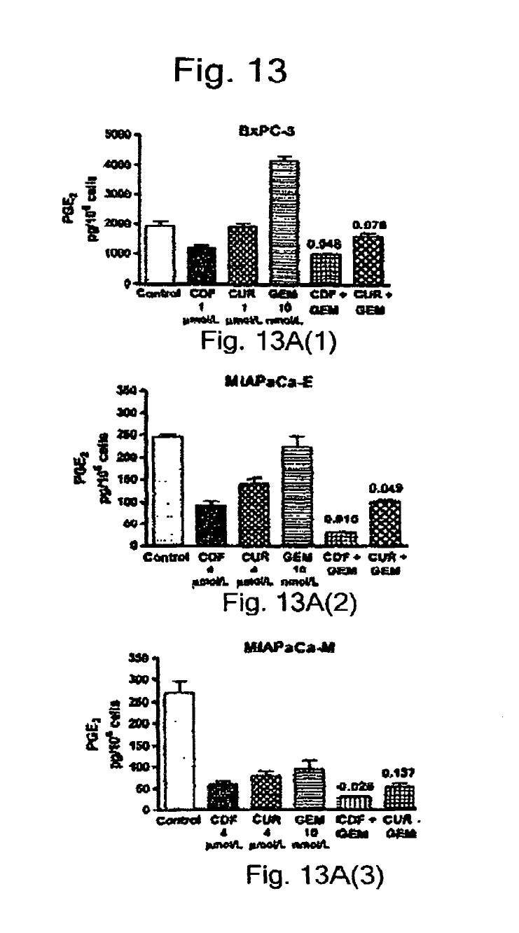

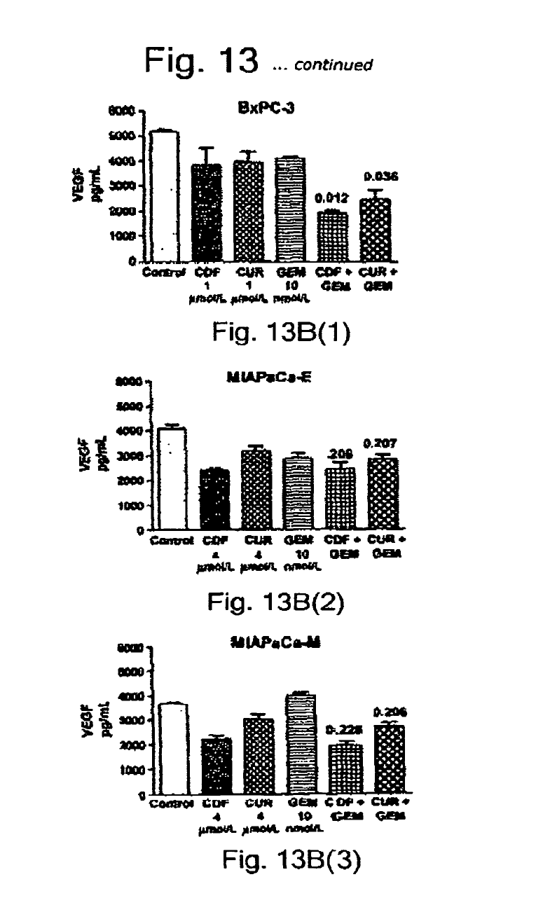

As demonstrated in the EMSA assay shown in FIG. 13, CDF can inhibit COX-2. Since it is known that the inhibition of COX-2 will reduce the synthesis of PGE₂, the levels of PGE₂ was measured in conditioned medium collected from BxPC-3, MIAPaCa-E, and MIAPaCa-M cells after treatment with CDF or CUR (1-4 μMol/L), GEM (10 nMol/L), or the combinations, for 24 h in serum-free media. PGE₂ secreted in the culture medium was analyzed using a PGE₂ immunoassay kit, as suggested by the manufacturer ®& D Systems, Minneapolis, Minn.).

Referring to FIG. 13A, there was a higher level of PGE₂ secretion by BxPC-3 cells as compared to MIAPaCa-E and MIAPaCa-M cells. The MIAPaCa-E and MIAPaCa-M cells showed very low basal levels of secreted PGE₂ which is consistent with low constitutive expression of COX-2 protein. There was a substantial increase in the level of PGE₂ secretion in GEM treated BxPC-3 cells as compared to untreated cells. This suggests that GEM-induced NF-κβ could be responsible for the induction of COX-2, which consequently induces secretion of PGE₂. A significant reduction in PGE₂ level was observed in cells treated with CDF, CUR, or their combinations with GEM; however, the effect was much more pronounced in cells treated with CDF than with CUR. Collectively, these results suggest that the production of PGE₂ is mediated through the NF-κβ and COX-2 pathway, and that CDF can down-regulate both NF-κβ and COX-2 thereby reducing the synthesis and secretion of PGE₂.

6. Inhibition of VEGF Secretion

The effect of CDF on another downstream transcriptional target of NF-κβ, vascular endothelial growth factor (VEGF) secretion, was studied in the three human pancreatic cell lines. Conditioned media, following a 72 h treatment with CDF, CUR, (1-4 μMol/L), GEM(10 nMol/L), or their combinations, was analyzed using an AlphaLISA VEGF 500 data point kit (Perkin Elmer, Norton, Ohio). Compared to untreated cells, and cells treated with CUR alone, CDF showed significant inhibition of secreted levels of VEGF in both MIAPaCa-E and MIAPaCa-M cell lines, as shown in FIGS. 13B(1) to FIG. 13B(3).

7. Modulation of miR-200b and miR-200c Expression

Previous studies have shown that loss of the miR-200 family is associated with the drug-resistant phenotype, especially in those cells having mesenchymal phenotype. Accordingly, the levels of expression of miR-200 in the three PC cell lines, after exposure to treatment, were investigated to gain further insight into the mechanisms of the biological effects of CDF.

The expression of miRNAs (miRNA-200a, miR-200b, and miR-21) in all three pancreatic cancer cell lines, was measured with a TaqMan MicroRNA Assay kit (Applied Biosystems, Carlsbad, Calif.) following the manufacturer's protocol. Cells were treated with CDF or CUR (1-4 μMol/L), GEM (10 nMol/L), or the combinations, for 72 h. Total RNA was extracted, and 5 ng of each sample were reverse-transcribed in a method described by Li, et al., Cancer Res., Vol. 69, pages 6704-6712 (2009). Real-time RT-PCR reactions were then carried out in a total volume of 25 μl reaction mixture, as described by Li, et al., id., using Smart Cycler II (Cepheid, Sunnyvale, Calif.). The data were analyzed using Cₜ method and were normalized by RNU6B expression in each sample. The results are shown in FIG. 14.

Referring to FIGS. 14A(1) and 14A(2), the level of expression of miR-200b and miR-200c, which are known regulators of EMT, were significantly suppressed in both GEM-resistant cell lines MIAPaCa-E and MIAPaCa-M, as compared to the GEM-sensitive cell line BxPC-3. This is consistent with their differential sensitivities to GEM. While BxPC-3, MIAPaCa-E, and MIAPaCaM cells treated with CDF, CUR, or GEM, and their combinations, all showed increased expression of miR-200b and miR-200c, the effect was much more pronounced for the CDF-treated cells, as shown in FIGS. 14B and 14C. This demonstrates that CDF is superior to the naturally-occurring CUR. These studies further indicate that the phenotypic characteristics of the GEM-resistant cell lines MIAPaCa-E and MIAPaCa-M, which also have EMT characteristics, can be reversed by CDF treatment so as to restore gemcitabine chemo-sensitivity.

The synthetic analog of curcumin, CDF, is significantly more effective at killing gemcitabine-resistant cells than naturally-occurring curcumin. This could be, in part, due to better cellular uptake and retention of CDF by PC cells, as shown by the pharmacokinetic data in mice reported hereinabove.

While not wishing to be bound by theory, the mechanism of action may involve inactivation of NF-κβ which in turn inactivates the transcription of COX-2, and thereby inhibits the production of PGE₂. The expression and activation of COX-2 and NF-κβ pathways are common in PC cells, and contribute to the observed resistance of PC cells to chemotherapeutic agents. CDF, either alone or in combination with GEM, is also more effective in inhibiting PGE₂ and VEGF. COX-2 generated PGE₂ plays an important role in pancreatic tumorigenesis. The data presented herein clearly shows that CDF, alone or in combination with GEM, inhibits the production of PGE₂ which may be useful for the prevention of tumor progression and/or treatment of PC.

Both CDF and curcumin are effective in reducing specific miRNAs. Advantageously, MiRNAs are moderately stable, as compared to large molecules, such as proteins, and can be efficiently extracted because they are well preserved in both formalin-fixed and paraffin-embedded tissues. MicroRNAs can also normalize multiple coding genes associated with tumor growth, and thus assessment of specific miRNA expression is useful for predicting disease outcome. It is well know that the development of cancer involves alterations in the expression of multiple genes regulated by transcriptional, post-transcriptional, translational, and post-translational modification, and, therefore, expression of a single gene, or protein, cannot accurately reflect the status of the disease. However, miR-21 is over-expressed in many solid tumors and has been shown to be associated with tumor progression, poor survival rates, and reduced effectiveness of known therapies. Expression of miR-21 is up-regulated in gemcitabine-resistant cell lines, such as MIAPaCa-E and MIAPaCa-M, compared to gemcitabine-sensitive BxPC-3 cells. The expression of miR21 could be significantly down-regulated by administering CDF or the combination of CDF and gemcitabine. There are several genes that are regulated by miR-21. The increased expression of miR-21 could down-regulate specific genes, one of which is PTEN, a well known tumor suppressor gene. The studies reported hereinabove show that CDF or CUR re-activate PTEN in PC cells. Consistent with these results, inactivation of miR-21 by treatment of cells with the anti-sense oligonucleotide of miR-21 increased PTEN protein expression and induced cell cycle arrest in human pancreatic cancer cells.

The implication of this is enormous because naturally-occurring, non-toxic agents, such as CUR, or its synthetic analogs, could be used to up-regulate PTEN, mediated via inactivation of miR-21. The data presented herein suggests that this strategy could sensitize drug-resistant PC cells to conventional cytotoxic agents, such as gemcitibine, which are not very effective by themselves. The activation of PTEN by CDF or curcumin can decrease Akt phosphorylation, as shown in FIG. 11, action can contribute to the inhibition of cell growth and induction of apoptosis.

The data presented on miR-21 inactivation is in sharp contrast to the data on miR-200b and miR-200c whose expression was drastically reduced in GEM-resistant PC cells. This is consistent with previous findings showing that the expression of these miRNAs was either lost, or substantially reduced, in various tumors, including pancreas tumors. In the studies reported hereinabove, the expression of miR-200b and miR-200c was up-regulated by CDF and CUR, suggesting that the mesenchymal phenotype of gemcitabine-resistant PC cells could be reversed by simply treating the cells with either CDF or CUR. Both GEM-resistant cell lines cells showed lower expression of both Mir-200b and Mir-200c, and loss of E-cadherin expression, which is consistent with the mesenchymal-like morphology shown on FIGS. 10A(2) and 10A(3) compared to BxPC-3 cells having epithelial morphology (FIG. 10A(1)) and higher expression of both Mir-200b and miR200c and E-Cadherin. Treatment of MIAPaCa-E and MIAPaCaM cells with CDF, alone or in combination with GEM, significantly up-regulated Mir-200 expression. The expression of E-cadherin was not up-regulated, suggesting that only partial reversal of the EMT could be achieved. Nevertheless, this could still lead to sensitization of gemcitabine-resistant cells to be subject to gemcitabine-induced killing.

III. Method of Treating Pancreatic Cancer

The experiments reported herein support the use of the curcumin analogs of the present invention, and in a particularly advantageous embodiment, the use of CDF to sensitize PC cells to gemcitabine by inactivation of NF-κβ, COX-2, and their downstream target molecules, which is in part due to inactivation of miR-21, and re-activation of miR-200b and miR-200c. The inactivation of miR-21 leads to the re-activation of PTEN, resulting in the inactivation of phosphorylated Aκt. In addition, CDF-induced re-activation of miR-200b and miR-200c, may in turn result in the reversal of EMT phenotype, thereby restoring sensitivity PC cells that have already become sensitive to gemcitabine.

Therefore, the invention includes a method of sensitizing drug-resistant pancreatic cancer cells, and in particular, gemcitabine-resistant pancreatic cancer cells to gemcitabine, for the prevention of tumor progression and/or treatment of pancreatic tumors in a subject who has been diagnosed with pancreatic cancer.

The method includes administering to the subject, who has been diagnosed with pancreatic cancer, a therapeutically effective amount of a fluorinated curcumin analog in accordance with the invention, and in a particularly advantageous embodiment, CDF, or a combination of CDF and the drug to which the cancer cells have become resistant. In the particular embodiment studied herein, the drug is gemcitabine. Since it has been demonstrated that CDF has the ability to restore sensitivity, in addition to its other beneficial effects through other paths of action, the use of the novel curcumin analogs, and particularly CDF, in the practice of the method herein may prevent tumor progression and facilitate the treatment of pancreatic tumors, that may, or may not, have developed gemcitibine-resistance.

Of course, while the method has been directed to pancreatic tumors, and to gemcitibine-resistance, it is to be understood that the principles of the invention would apply to other cancers and other chemotherapeutic agents. In addition, the interpretation of the invention is not to be limited by, modification of the mechanistic pathways described in the specific example above, i.e., inactivation of NF-κμ, COX-2, and their downstream target molecules, etc.

As an example, emerging evidence suggests that drug-resistance in tumor cells is due to tumor initiating cells, or CSCs, in the tumor mass. CSCs, which have the capacity for self-renewal and the potential to regenerate into all types of differentiated cells, giving rise to heterogeneous tumor cell populations in a tumor mass, and contribute to tumor aggressiveness. The following studies were conducted in order to assess whether CDF, CUR, or combinations of CDF and CUR with other cytotoxic agents, such as gemcitibine, can target CSCs in vitro and in vivo.

IV. Use of CDF to Inhibit Pancreatic Cancer CSCs

CDF significantly inhibits the sphere-forming ability of PC cells to create pancreatospheres. The studies reported in this Section show increased disintegration of pancreatospheres, which is associated with attenuation of the CSC markers, CD44 and EpCAM, particularly in GEM-resistant MIAPaCa-2 PC cells. GEM-resistant MIAPaCa-2 PC cells contain a high proportion of CSCs, which is consistent with increased MIr-21 expression and decreased MIr-200 expression.

In a xenograft mouse model of human PC, CDF treatment significantly inhibited tumor growth, which was associated with decreased NF-κβ DNA binding activity, COX-2, and miR-21 expression, and increased PTEN and miR-200 expression in tumor remnants. Thus, the anti-tumor activity of CDF appears to be strongly associated with inhibition of CSC function via down-regulation of CSC-associated signaling pathways.

1. Creation of Drug-Resistant Pancreatic Cancer Cell Lines

Human pancreatic cancer cell lines AsPC-1, and MIAPaCa-2 were purchased from ATCC (Manassas, Va.). The two cell lines AsPC-1 and MIAPaCa-2 were exposed to 200 nmol/L of GEM and 5 μMol/L of tarceva (erlotinib) every other week for about 6 months to create gemcitabine- and tarceva-resistant cell lines (herein designated GTR), named as AsPC-1-GTR and MIAPaCa-2-GTR, respectively. AsPC-1 and MIAPaCa-2 cell lines and their clones were chosen because of their relatively resistant nature.

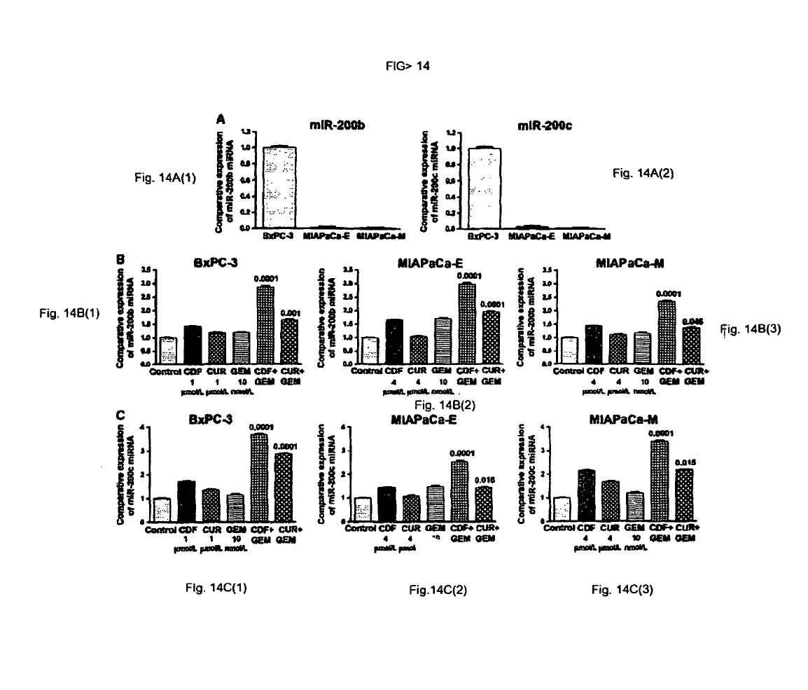

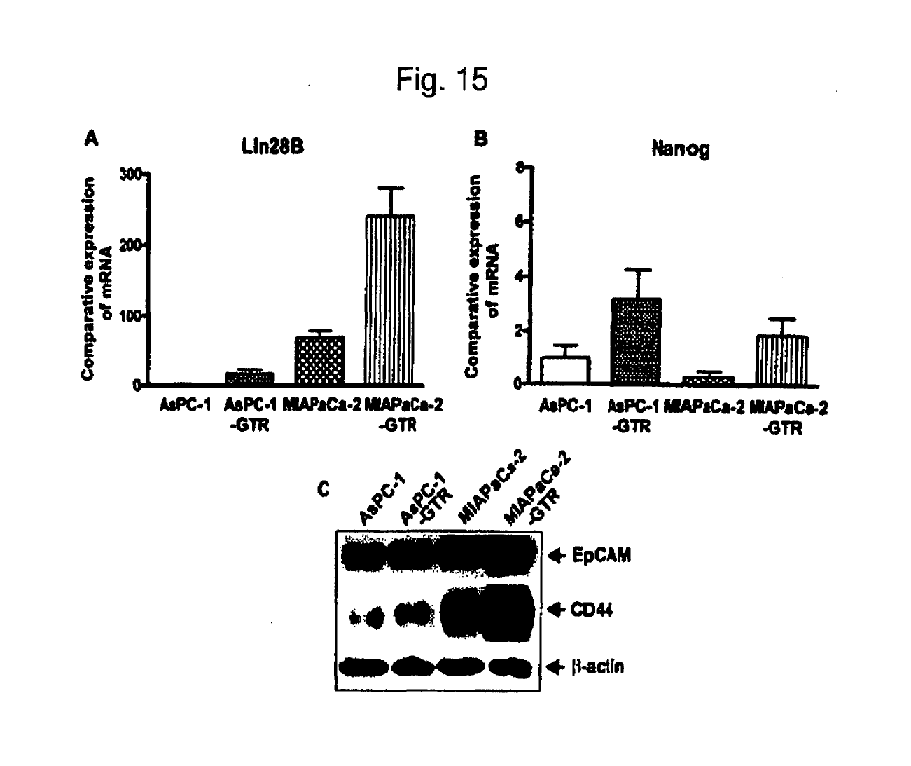

The CSC characteristics of the GTR cells was confirmed, using the stem cell markers, Lin28B and Nanog. Referring to FIGS. 15A and 15B, which are graphical representations of the comparative expression of Lin28B and Nanog mRNA, as assessed by qRT-PCR, the GTR cell lines exhibited increased expression as compared to the parental cell lines. Likewise, Western blot analysis (FIG. 15 C) showed increased expression levels of the CSC markers, EpCAM and CD44, in the PC-GTR cells, thereby supporting the CSC nature of the PC-GTR cell lines.

2. Evaluating the Use of CDF to Inhibit PC CSCs

(1) Clongenic Assay

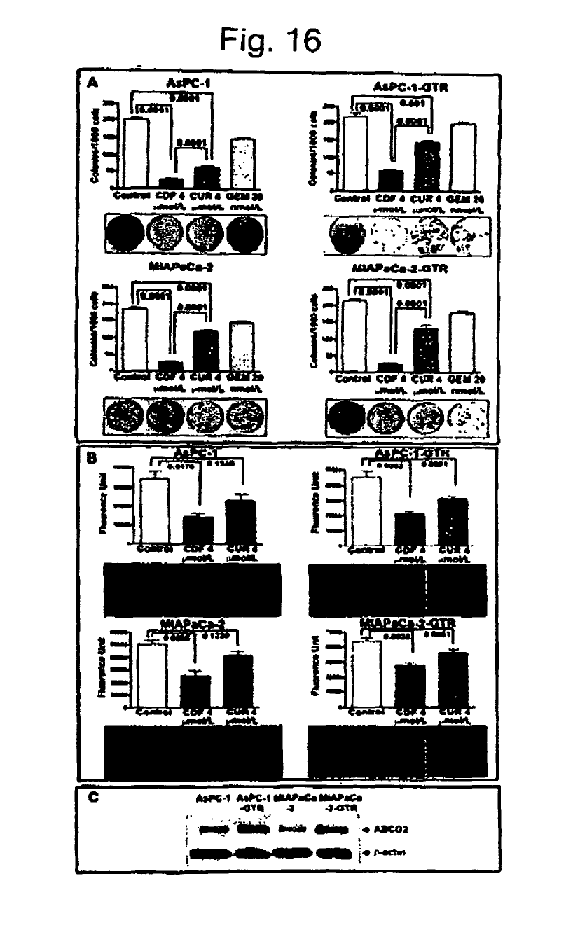

AsPC-1, MIAPaCa-2, AsPC-1-GTR and MIAPaCa-2-GTR cells were treated with 20 nmol/L of GEM and 4 μmol/L of CUR or CDF according to the clongenic assay procedure set forth in Section D(1)(2) above. Referring to FIG. 16A, there was a significant reduction in clonogenicity of AsPC-1 and MIAPaCa-2 cells treated with CUR and CDF, but not with GEM. However, CDF treatment had a much greater and significant reduction in colony formation as compared to CUR. AsPC-1-GTR and MIAPaCa-2-GTR cells had an 80% reduction of clonogenicity with CDF treatment, whereas, there was only a 20-30% reduction of clonogenicity observed with GEM or CUR treatment. Overall, CDF treatment showed a significant reduction in clonogenicity of human PC cells, once again demonstrating the superiority of CDF for treating and/or preventing progression or recurrence of cancer.

(2) Invasion Assay

The invasive activity of the drug resistant cell lines was tested by using a BD BioCoat Tumor Invasion Assay System (BD Biosciences, Bedford, Mass.) according to the manufacturer's protocol. Cells (5×10⁴) were seeded and maintained in serum free medium supplemented with CUR or CDF. The fluorescence was read using a TECAN Microplate Reader at 530/590 nm and photographed. Referring to FIG. 16B, both CDF and CUR treatment decreased PC cell migration and invasion. However, 4 μmol/L CUR had minimal inhibition on invasion whereas CDF, at a similar concentration, showed significant inhibition of invasion, thereby demonstrating once again its superior action.

There was a basal level of ABCG2 expression in the parental cell lines (de novo drug-resistant cells); however, the level of expression of ABCG2 was increased in the GTR cells (acquired drug resistance) according to the chromatograph shown in FIG. 16C.

(3) MTT Assay

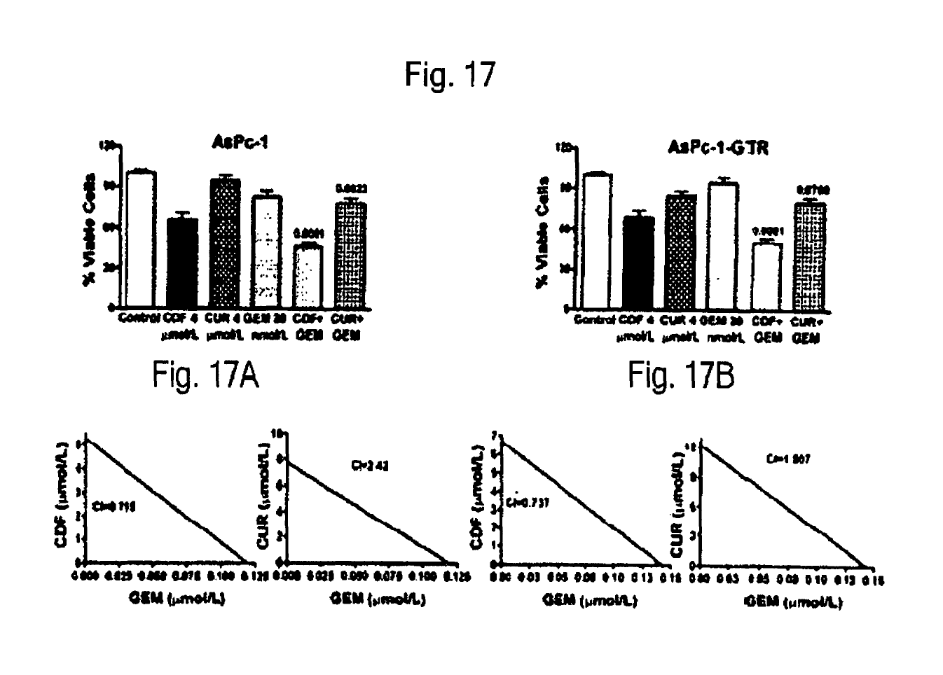

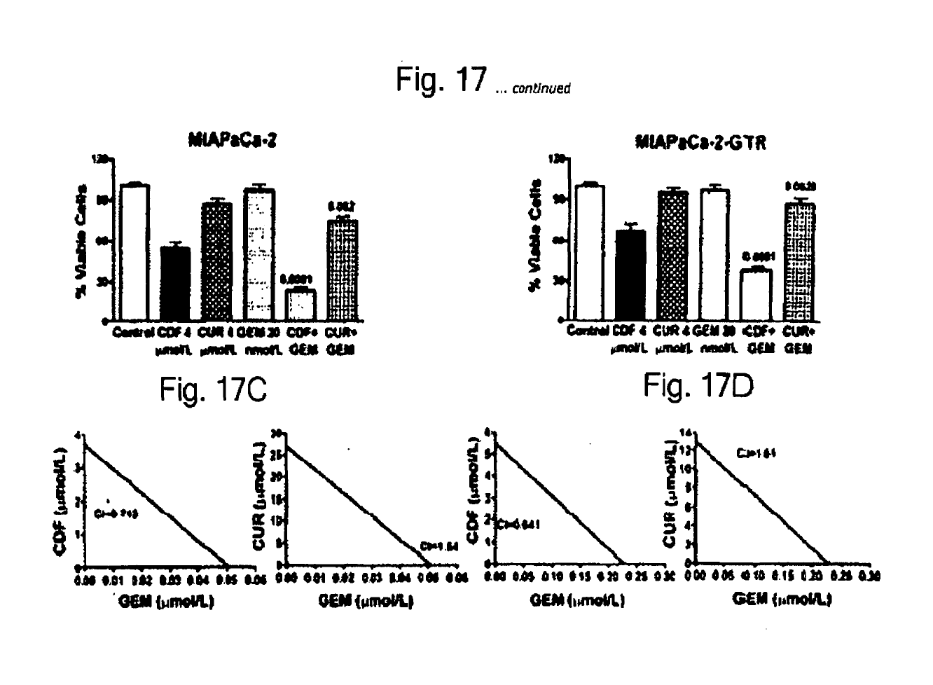

An MTT assay was conducted to study the cell survival after 72 h of treatment of all four cell lines with CUR (4 μMol/L), CDF (4 μMol/L), GEM (20 nMol.L) and combinations of CDF/GEM and CUR/GEM GEM. Untreated control has been assigned a value of 100%. The p value shown represents comparisons between a single agent t and their combinations by using a paired t-test. A combination Index (CI)<1 for the CDF and GEM combination indicates synergism. The results are shown on FIGS. 17A to 17D.

CDF, particularly in combination with GEM, caused a remarkable reduction of cell survival in all four cell lines as compared to the combination of CUR and GEM. Furthermore, analysis of drug combination treatment showed that the combination index after treatment with CDF/GEM was less than 1.00, suggesting a synergistic effect. In contrast, the combination index with CUR/GEM was more than 1.00, showing a non-synergistic effect. Overall, these results show that CDF caused a much more significant reduction of cell survival in PC cells than GEM, CUR, or their combination.

(4) Sphere Disintegration Assay

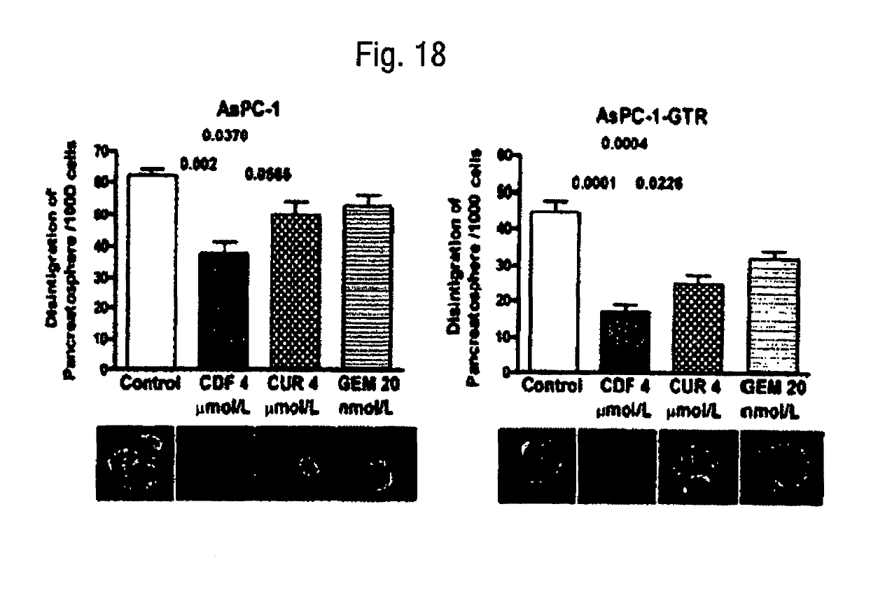

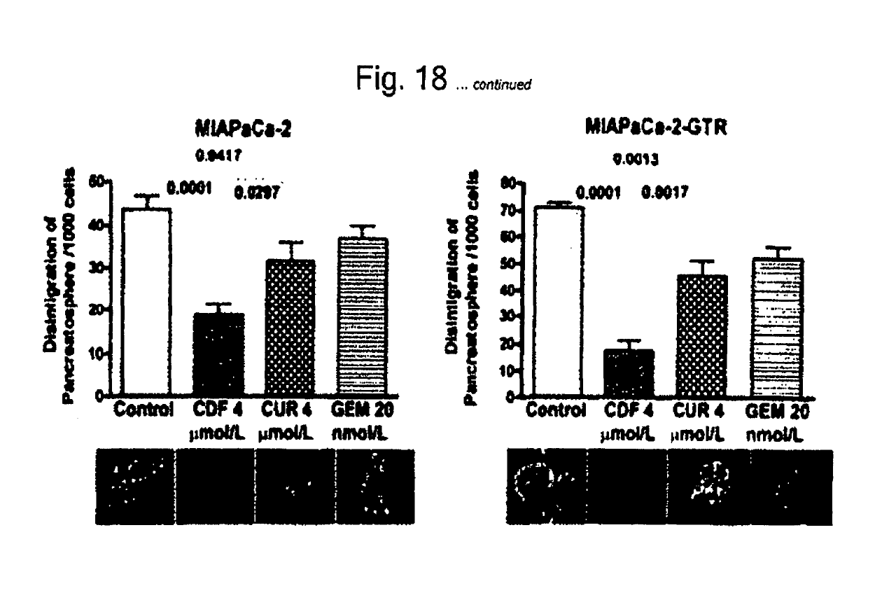

An assay was conducted to examine the effect CUR, CDF, and GEM on the ability of the four PC cell lines to form pancreatospheres, and the disintegration of formed pancreatospheres. After allowing 10 days to generate the formation of pancreatospheres, the cells were subjected to the treating agent for 5 days. Referring to FIG. 18, CDF remarkably increased disintegration of pancreatospheres in AsPC-1, AsPC1-GTR, MIAPaCa-2, and MIAPaCa-2-GTR cells.

(5) Sphere Formation Assay

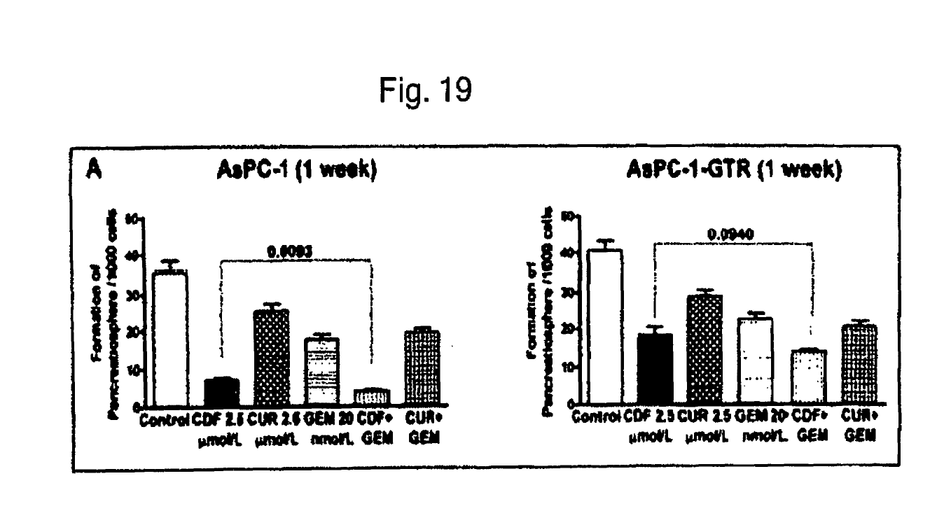

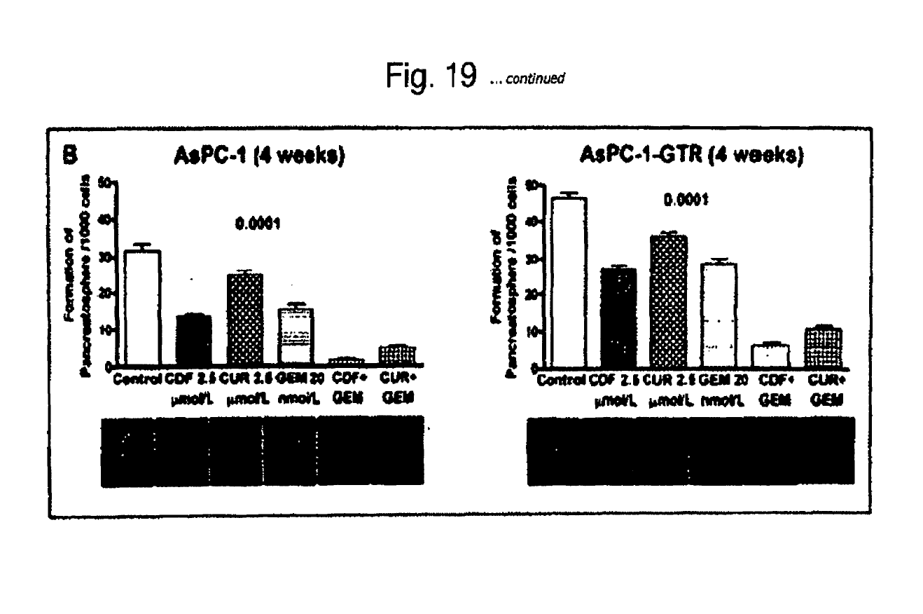

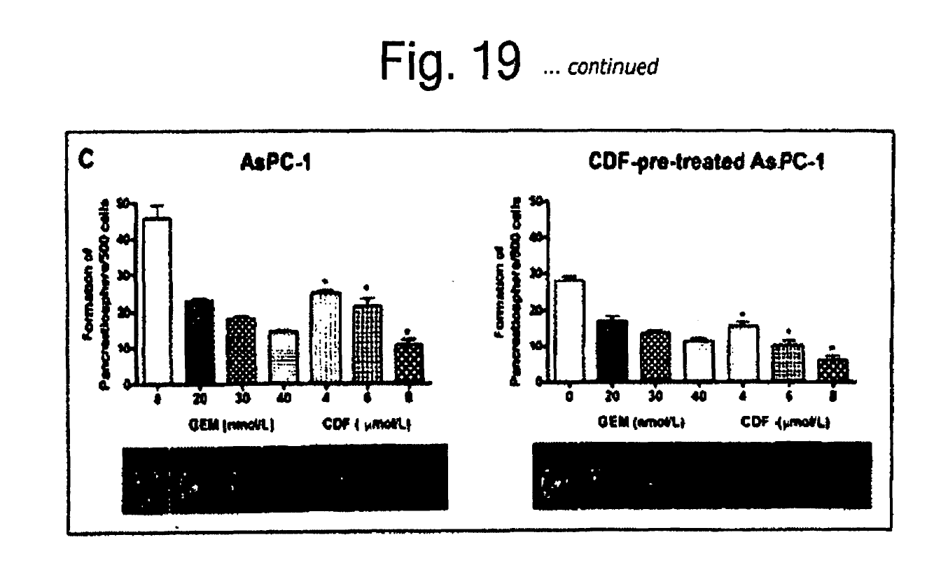

A sphere formation assay was conducted for 1 week (FIG. 19A) and four weeks (FIG. 19B) to examine the effect of the treating agents on the CSC self-renewal capacity of the PC cell line AsPC-1. Referring to FIG. 19A, CDF/GEM completely eliminated the formation of pancreatospheres after four weeks of treatment. This was significantly better than the effect of CUR or the combination of CUR/GEM, and occurred even in GEM-resistant PC cells. Therefore, it appears that CDF may cause pancreatospheres to be more sensitive to GEM, and as a result, CDF, or the combination of CDF/GEM, would be useful for targeted killing of CSCs.

FIG. 19C shows the effect of different concentrations of GEM and CDF on 2^{nd} passage of pancreatospheres in pre-treated primary pancreatospheres created by AsPC-1 cells. CDF treatment inhibited 2^{nd} passage of pancreatospheres in a dose-dependent manner. Furthermore, pretreatment with CDF improved the outcome.

(6) Expression of CD44 and EpCAM

Single cell suspensions of AsPC-1 and AsPC-1-GTR were plated into sphere formation media as previously described. After 7 days, the pancreatospheres were collected, washed, and fixed. The CSC biomarkers, CD44 and EpCAM, were used for immunostaining and the pancreatospheres were examined with confocal microscopy. CDF decreased the expression of CD44 and EpCAM in pancreatospheres, suggesting that the inhibitory effect of CDF on pancreatosphere formation may be associated with the inhibition of CD44 and EpCAM expression.

3. Inhibition of PC Tumor Growth In Vivo

(1) Growth of MIAPaCa-2 Mouse Xenograft Tumors

A subcutaneous xenograft tumor model was used to evaluate the in vivo effect of CDF, CUR, GEM, and the combinations CDF/GEM and CUR/GEM on MiAPaCA-2 tumors. Female CB17 SCID mice 4 wks old were purchased from Taconic Farms (Germantown, N.Y.). Small fragments of the MIAPaCa-2 xenograft were implanted subcutaneously and bilaterally into mice. Once the mice developed palpable tumors, they were randomly selected for the following treatment groups (n=5/group): (1) untreated control; (2) CDF (5 mg/mouse/day), intragastric once daily for 12 days; (3) CUR (5 mg/mouse/day), intragastric once daily for 12 days; (4) GEM (1 mg/mouse/day), intravenous every third day for a total of three doses; (5) CDF/GEM gemcitabine using the doses indicated above; (6) CUR/GEM using the doses as indicated above. Tumor measurements and changes in weight were performed and tissue was stored at −70° C. for RNA and protein extraction.

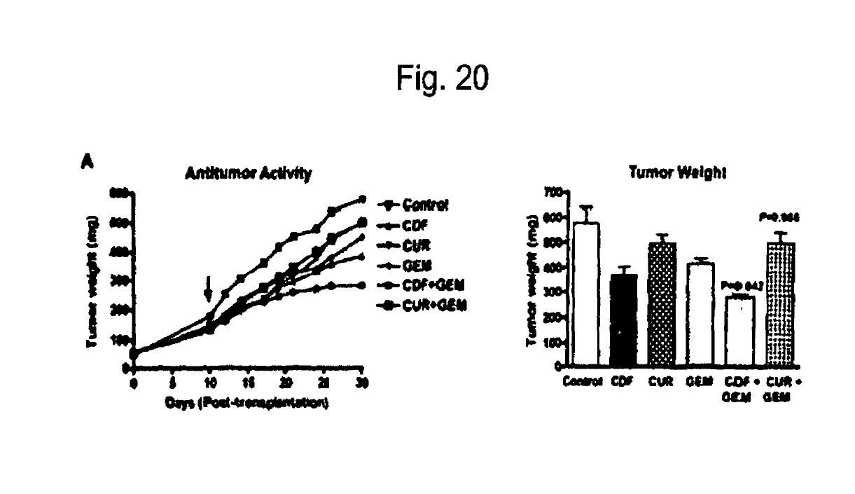

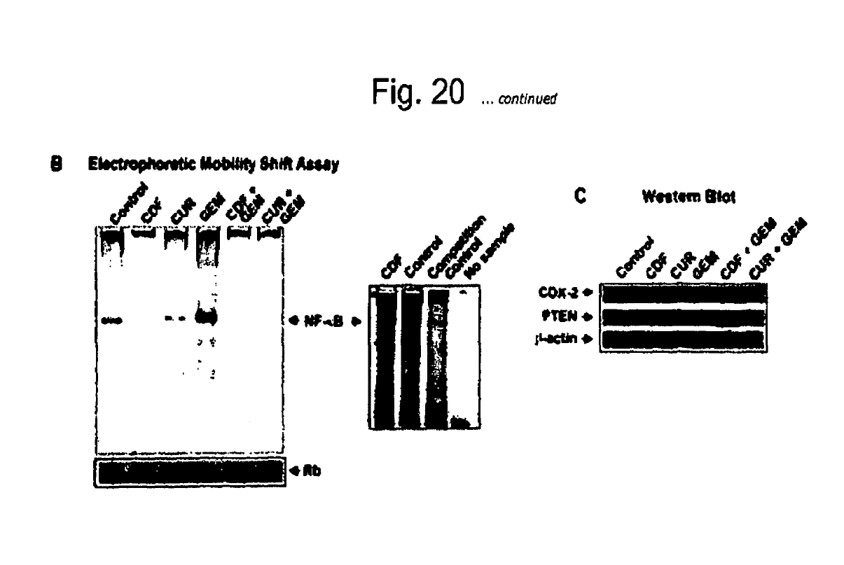

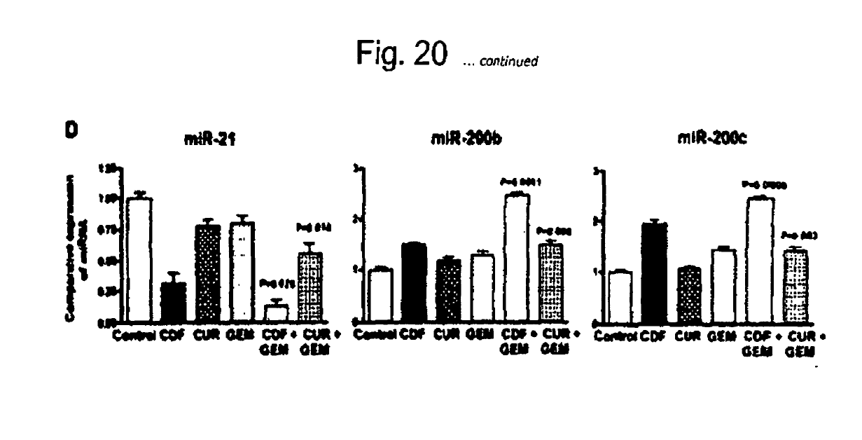

CDF/GEM treatment significantly inhibited tumor growth in MIAPaCa-2 tumors as compared to CUR/GEM as shown in FIG. 20A. Referring to FIG. 20A, the arrow represents the day that treatment was initiated. The mice did not show any appreciable weight loss during the 30 day treatment period showing that these treatments had no major adverse effects on animals.

(2) EMSA for NF-κB DNA Binding Activity in PC Cells In Vivo

Nuclear extracts were prepared from tumor tissue induced by the MIA PaCa-2 cells using an homogenizer with 400 μl of ice cold lysis buffer extracted as described earlier. EMSA was performed using the Odyssey Infrared Imaging System with NF-κβ 1RDye labeled oligonucleotide from LI-COR, Inc. (Lincoln, Nebr.). An NF-Kβ competition control study was conducted using unlabeled NF-Kβ consensus oligonucleotide. The samples were loaded and run at 30 mA for 1 hour. The gel was scanned using Odyssey Infrared Imaging System (LI-COR, Inc.).

The tumor tissues were treated with CDF, CUR, GEM and the CDF/GEM and CUR/GEM combinations. Both CDF and CUR down-regulated NF-κβ activation whereas GEM activated NF-κβ levels, which effect was abrogated in the combination treatment CDF/GEM. The combination treatment CDF/GEM showed a significant decrease in NF-κβ level as compared to CUR/GEM (See, FIG. 20B), suggesting that inactivation of NF-κB could be one of the molecular mechanisms by which CDF elicits its anti-tumor activity against PC tumors.

(3) Protein Expression In Vivo

COX-2, PTEN, and β-actin expression was determined by Western blot. A significant down-regulation in the expression of COX-2 was observed after treatment with CUR/GEM and CDF/GEM, however, the effect was more pronounced for CDF/GEM. The expression of PTEN, a tumor suppressor gene, was found to be decreased in MIAPaCa2 cells; however, the expression of PTEN was up-regulated after treatment with CDF (FIG. 20C). These results suggest that CDF is much more effective than CUR.

(4) Modulation in Expression of MiR-21 and MiR-200 Family In Vivo

PTEN, a known target of miR-21, has been reported to be up-regulated in PC. Therefore, the expression of miRNAs (miRNA-200b, miR-200c, and miR-21) in MIAPaCa-2 tumors, was assessed with a TaqMan MicroRNA Assay kit (Applied Biosystems, Carlsbad, Calif.) following the manufacturer's protocol. 5 ng of total RNA was reverse transcribed and real-time PCR reactions were carried as previously described. Over-expression of MiR-21 was observed in MICaPa-2 tumors whereas there was a significant reduction in the expression of MiR-21 in tumors treated with CDF and CDF/GEM as shown on FIG. 20D.

The expression levels of miRNA-200b and miR-200c, which are known regulators of EMT, were found to be significantly low in MIAPaCa-2 cells (FIG. 20D). In contrast, CDF and CDF/GEM showed increased expression of both miR-200b, and miR-200c, however, the effect with CUR or CUR/GEM was minimal. This further demonstrates the superiority of CDF in suppressing the expression of miR-21, resulting in the re-expression of PTEN and of miR-200, which could be responsible for the reversal of EMT phenotype in cells treated with CDF. Overall, these results show that the phenotypic characteristics of MIAPaCa-2 tumors are consistent with enriched population of CSCs and EMT characteristics, and that these drug-resistant cells can be killed CDF alone, or in combination with GEM.

4. Growth of CSC In Vivo

Pancreatospheres (5,000) derived from MIAPaCa-2 cells were isolated and implanted in mice with 1:1 matrigel. The growth rate was observed for a period of 30 days. RNA was extracted from the tumor tissue and assayed.

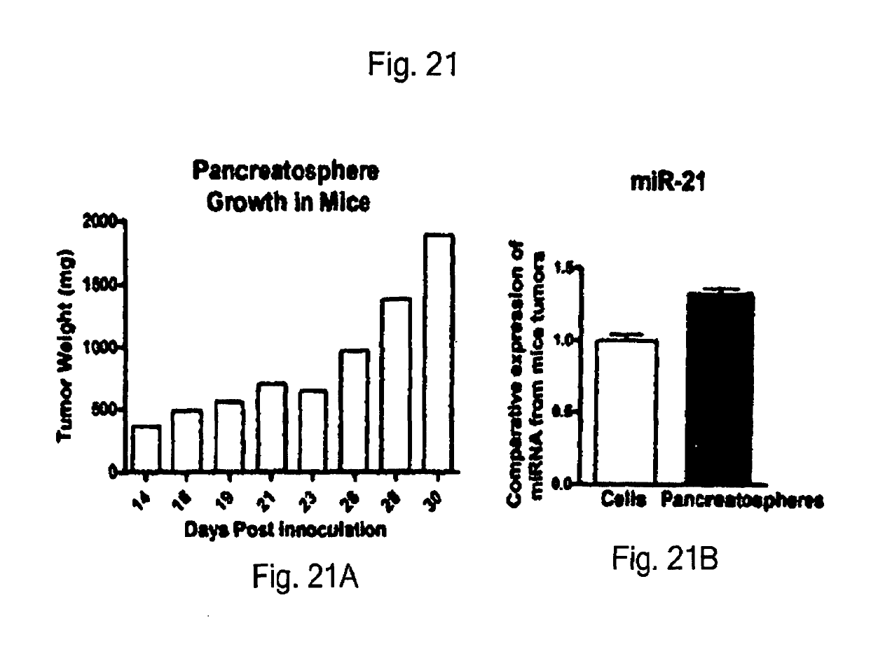

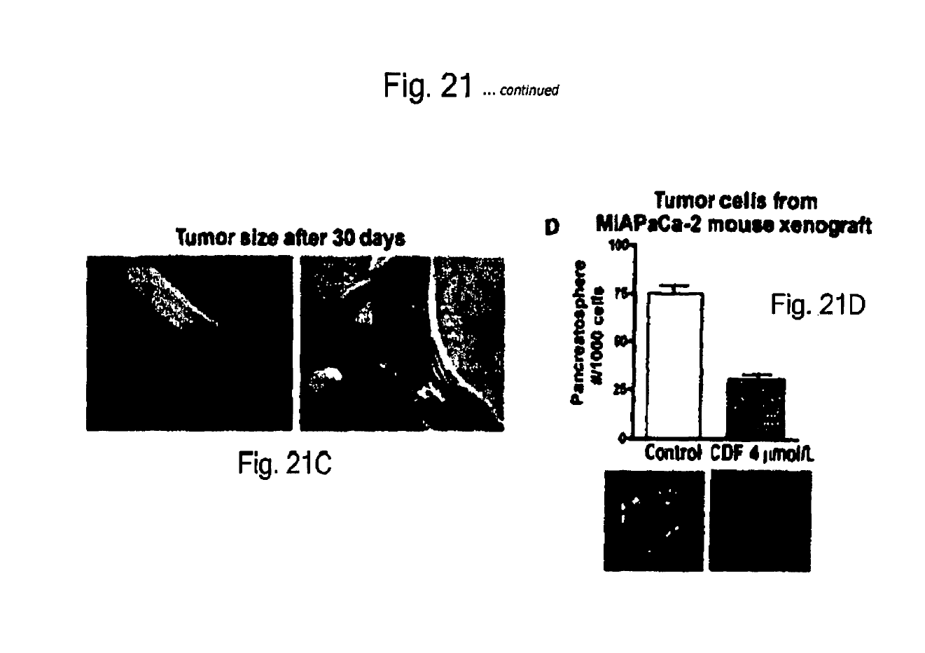

Tumor weight increased significantly as the days progressed post-inoculation. (FIG. 21A). There was a moderate increase in expression of miR-21, as measured by real-time RT-PCT, in tumors derived from the implanted pancreatospheres as compared to tumor\s implanted with one million parental MIAPaCa-2 cells (seem FIG. 21B).

The animal was euthanized after 30 days because of tumor burden and photographed. The tumors can be observed in FIG. 21C which shows the presence of larger tumors, as well as loco-regional lymph node metastasis, whereas tumors derived from the parental cells did not show any metastasis over a period of 30 days. Treatment of pancreatosphere-derived tumor cells with CDF resulted in significant inhibition in the formation of new pancreatospheres (FIG. 21D). These results show that CDF along, or in combination with GEM, can kill drug-resistant cells enriched in CSCs (pancreatospheres) in vivo.

V. Method of Treating of Pancreatic Cancer

In another method of treating embodiment, there is provided a method of treating pancreatic cancer and/or preventing the preventing the recurrence of pancreatic cancer tumors by administering a therapeutically effective amount of the fluorinated curcumin analog in accordance with the present invention, either alone or in combination with another one or more chemotherapeutic or cytotoxic agents, such as GEM, to a subject in need of treatment. The flurocurcumin analogs of the present invention inhibit growth and reoccurrence by targeting self-renewal pathways and/or reducing or eliminating CSCs. In a particularly preferred embodiment, the analog is CDF.

VI. Use of CDF to Inhibit Colon Cancer CSCs

In addition to the foregoing, studies have been conducted that demonstrate that the analogs of the present invention, and particularly CDF, can be used as anti-tumor agents in the prevention and/or treatment of colon cancer. In particular, the effectiveness of CDF and CUR was evaluated in inhibiting the growth of 5-FU+Ox-resistant cancer cells.

In studies that mimic those reported hereinabove with respect to pancreatic cancer, the formation and disintegration of colonospheres, which are enriched in colon cancer CSCs, was observed. The results of these studies confirmed that CDF, either alone or in conjunction with conventional chemotherapy, such as 5-FU+Ox or FOLFOX, is superior to naturally-occurring CUR in reducing and/or reversing chemoresistant colon cancer cells, and is accompanied by inhibition of growth, induction of apoptosis, and disintegration of colonspheres. These changes were associated with don-regulation of the membrane transport ABCG2 and attenuation of EGFR, IGF-IR, and NF-κB signaling, consistent with inactivation of β-caterin, COS-2, c-Myx, and Bxl-xL and activation of the pro-apoptotic Bax.

The studies leading to this conclusion can be found in Kanwar et al., Pharm. Res., Difluorinated-Curcumin (CDF): A Novel Curcumin Analog is a Potent Inhibitor of Colon Cancer Stem-Like Cells, Pharm. Res., (Epub: Dec. 14, 2010), the disclosure of which is incorporate herein by reference.

Although the studies reported herein has been addressed to the use of the analog CDF, it is to be understood that other analogs falling within Formula I, are intended for use in the methods of the present invention.

Although the invention has been described in terms of specific embodiments and applications, persons skilled in the art may, in light of this teaching, generate additional embodiments without exceeding the scope or departing from the spirit of the invention described and claimed herein. Accordingly, it is to be understood that the drawing and description in this disclosure are proffered to facilitate comprehension of the invention, and should not be construed to limit the scope thereof.

1. The chemical compound of Formula 1: 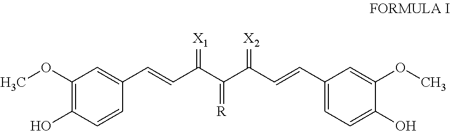
wherein X₁ and X₂ are the same or different and are selected from the group consisting of O, —OH, and an amino;
R is H or 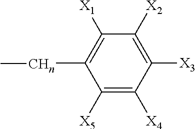
wherein x₁-x₅ are selected from the group consisting of —H, —F, —CF₃, —CHF₂, and CHₙ is a saturated or unsaturated lower alkyl where n=1-4 and wherein there are at least two fluoride atoms between X₁, X₂, and R.

2. The compound of claim 1 wherein the amino is selected from the group consisting of saturated and unsaturated, substituted and unsubstituted amines, alkylamines, arylamines, heterocyclic amines, phenylamines, naphthoylamines, isothiocyanates, semicarbazides, thiosemicarbazides, hydrazones, hydrazides, thioureas, hydroxamates, arylazos, azocylics, and carboxyamidrazones.

3. The compound of analogs of claim 1 wherein R═H or —CH₂—C₆H₄F₂.

4. The compound of claim 1 wherein X₁ and/or X₂, and preferably X₁═X₂, is thiosemicarbazide or semicarbazide of the general formula: 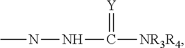
where Y═O or S and R₃ and R₄ are selected from the group

5. The compound of claim 1 wherein R is —CH₂—C₆H₄F₂.

6. The compound of claim 1 wherein X₁═X₂ and selected from the group consisting of O and an amino that forms a ligand that will conjugate with a metal ion to form a 1:1 metal ion to ligand complex.

7. A formulation comprising a therapeutically effective amount of a compound of Formula I, or a salt thereof, in a pharmaceutically-acceptable non-toxic carrier.

8. The formulation of claim 7 wherein the compound of Formula I is (1E,6E)-1,7-bis(4-hydroxy-3-methoxyphenyl)hepta-1,6-diene{4(3,4 difluorobenzaldehyde)}-3,5-dione.

9. The formulation of claim 7 further comprising at least one or more chemotherapeutic or cytotoxic pharmaceutical agents.

10. The formulation of claim 9 wherein the at least one or more chemotherapeutic or cytotoxic pharmaceutical agents are selected from the group consisting of genistein, celecoxib, gemcitabine, 5-flurouracil and oxaliplatin.

11. A method of preventing and/or treating cancer comprising administering a therapeutically effective amount of the analog of Formula I, or a pharmaceutically acceptable salt thereof, to a subject either alone, or in combination with, one or more chemotherapeutic and/or cytotoxic pharmaceutical agents.

12. A method of sensitizing drug-resistant pancreatic cancer cells for the prevention of tumor progression and/or treatment of pancreatic tumors in a subject who has been diagnosed with pancreatic cancer comprising the step of administering to the subject a therapeutically effective amount of a fluorinated curcumin analog of Formula I, either alone, or in combination with at least one other chemotherapeutic or cytotoxic pharmaceutical agent.

13. The method of claim 12 wherein the fluorinated curcumin is (1E,6E)-1,7-bis(4-hydroxy-3-methoxyphenyl)hepta-1,6-diene{4(3,4 difluorobenzaldehyde)}-3,5-dione.

14. The method of claim 13 wherein the at least one other chemotherapeutic or cytotoxic pharmaceutical agent is gemcitabine.

15. A method of inhibiting the growth of chemo-resistant colon cancer cells that are enriched in cancer stem-like cells in a subject having colon cancer comprising administering to the subject a therapeutically effective amount of a compound according to Formula I either alone, or in combination, with at least one other chemotherapeutic or cytotoxic pharmaceutical agent.

16. The method of claim 15 wherein the analog is (1E,6E)-1,7-bis(4-hydroxy-3-methoxyphenyl)hepta-1,6-diene{4(3,4 difluorobenzaldehyde)}-3,5-dione

17. The method of claim 16 wherein the at least one other chemotherapeutic or cytotoxic pharmaceutical agent is selected from the group consisting of 5-FU and/or Oxaliplatin, and FOLFOX.

18. The compound of claim 1 which is (1E,6E)-1,7-bis(4-hydroxy-3-methoxyphenyl)hepta-1,6-diene{4(3,4 difluorobenzaldehyde)}-3,5-dione: [chemical structure]
